(19) United States
(12) Patent Application Publication
(10) Pub. No.: US 2014/0296086 A1
(43) Pub. Date: Oct. 2, 2014

(54) SYSTEM AND METHOD FOR CELL-TYPE SPECIFIC COMPARATIVE ANALYSES OF DIFFERENT GENOTYPES TO IDENTIFY RESISTANCE GENES

(71) Applicant: Vincent P. Klink, Starkville, MS (US)

(72) Inventor: Vincent P. Klink, Starkville, MS (US)

(73) Assignee: MISSISSIPPI STATE UNIVERSITY, Mississippi State, MS (US)

(21) Appl. No.: 13/993,886

(22) PCT Filed: Dec. 13, 2011
(86) PCT No.: PCT/US2011/064743

§ 371 Date: Oct. 23, 2013

Related U.S. Application Data
(60) Provisional application No. 61/459,481, filed on Dec. 13, 2010.

(51) Int. Cl. C12Q 1/68

(52) U.S. Cl. CPC C12Q 1/6895
USPC 506/9

(57) ABSTRACT

Glycine max L. Merr. (soybean) resistance to Heterodera glycines Ichinohe is classified into two cytologically-defined responses, the G. max [Peking]- and G. max_{[PI 88788]}-types. Microarray analyses comparing these cytologically and developmentally distinct resistant reactions reveal differences in gene expression in pericycle and surrounding cells even before infection. Gene pathway analyses compare the two genotypes (1) before, (2) at various times during, (3) constitutively throughout the resistant reaction and (4) at all time points prior to and during the resistant reaction. The amplified levels of transcriptional activity of defense genes may explain the rapid and potent reaction in G. Max_{[Peking/PI 548402]} as compared to G. max_{[PI 88788]}.

### CROSS-REFERENCES TO RELATED APPLICATIONS

This application claims the benefit of U.S. Provisional Application No. 61/459,481 filed Dec. 13, 2010, incorporated herein by reference in its entirety.

### STATEMENT REGARDING FEDERALLY SPONSORED RESEARCH OR DEVELOPMENT

Not Applicable

### FIELD OF THE INVENTION

This invention is generally related to a system and method for cell-type specific comparative analyses of different genotypes to identify resistance genes.

### BACKGROUND OF THE INVENTION

The infection of plants by parasitic nematodes is a major, ubiquitous, dominant and persistent problem for agriculture, worldwide. Estimates rate worldwide agro-nomic losses to plant parasitic nematodes at 100-157 billion dollars, annually (Sasser and Freckman 1987; Abad et al. 2008). However, the ability to resist infection exists in the germplasm of many agricultural plants, including one of the most important, soybean (Glycine max). The dominant pathogen of G. max is the parasitic nematode Heterodera glycines (soybean cyst nematode; Wrather et al. 2001; Wrather and Koenning 2006). Many natural collections of G. max have been made, providing a bank of accessions (genotypes) that are catalogued by a plant introduction (PI) number. The numerous G. max accessions have been tested for their ability to resist infection by H. glycines (reviewed in Riggs 1992; reviewed in Shannon et al. 2004). From those studies, two major cohorts of PIs, each composed of a few G. max genotypes, were shown to exhibit specific but contrasting ways to combat H. glycines at the site of infection, a nurse cell known as a syncytium. The cohorts are each defined by their respective agronomically important archetypes, G. max Peking (G. max_{[}p_{eking]}) and G. max_{[PI 88788]} (G. max_{[PI 88788]}). Importantly, G. max_{[}p_{eking]} and G. max_{[PI 88788]} are the sources of greater than 95% of the resistance germplasm that is bred into commercial varieties (reviewed in Concibido et al. 2004).

The response of G. max_{[}p_{eking]} to H. glycines infection is characterized by a potent and rapid resistance reaction (Klink et al. 2009a). It is potent because, depending on the H. glycines population infecting G. max, the nematodes die at the parasitic second stage juvenile (p-J2). In contrast, G. max_{[PI 88788]} is characterized by a potent but prolonged resistant reaction (Klink et al. 2010a) where nematodes die at the J3 and J4 stages. An interesting feature of these resistant reactions, occurring at the syncytium, is that their underlying cytology is very different. While only a few molecular investigations have studied those contrasting forms of the resistant reaction (Klink et al. 2007a, b, 2009a, 2010a, b), they have never been directly compared to each other until now. Thus, it has not been determined how a single nematode population can elicit the development of two completely different resistant reactions at the site of infection.

In the studies presented here, the H. glycines population, HG-type 7, was used to infect both the G. max_{[PI 548402]} genetic background of the genotype Peking (G. max_{[Peking/PI 548402]}) and G. max_{[PI 88788]}. The experiments determined the gene expression pattern as a single pure nematode population elicits two completely different cellular responses culminating in resistant reactions in (G. Max_{[Peking/PI 548402]}) and G. max_{[PI 88788]}.

What makes these experiments important is that very few studies in agriculturally important plant systems have identified genes that relate to or confer resistance to parasitic nematodes. For example, Cai et al. (1997) identified a resistance (R) gene in Beta procumbens (sugar beet) that yields an incompatible reaction to the beet cyst nematode Heterodera schachtii. Milligan et al. (1998) identified the R gene, Mi, in Lycopersicon esculentum (tomato) to the giant cell-forming plant parasitic nematode Meloidiogyne incognita. The Mi gene has been shown to encode a leucine rich repeat (LRR) protein, a family of proteins with a long history of being involved in plant defense (Jones et al. 1994). Other genes that are involved in the resistance process to plant parasitic nematodes have also been identified. For example, Gao et al. (2008) identified the 9-lipoxygenase (ZmL0X-3) gene of Zea mays (corn), responsible for resistance to M. incognita. The work of Gao et al. (2008) implicates jasmonic acid signaling and lipid metabolism in defense to plant parasitic nematodes. LOX is the most highly induced gene, locally within syncytia undergoing an incompatible reaction as compared to the syncytia undergoing a compatible reaction in G. max (Klink et al. 2007a, 2009a). Other genes of the LOX signaling pathway have also been shown to be induced (Klink et al. 2009a, 2010a).

Glycine max currently is the top rated export crop in the US and the source of 70% (157 million metric tons) of the world's protein meal. Decades of gene mapping studies have been done to identify those resistance loci in G. max (reviewed in Concibido et al. 2004). The genetic mapping investigations reveal the resistance of G. max to H. glycines is multigenic, composed of both recessive and dominant genes (reviewed in Concibido et al. 2004). The recessive genes are rhg1, rhg2 and rhg3 (Caldwell et al. 1960). The two dominant resistance genes are Rhg4 (Matson and Williams 1965) and Rhg5 (Rao-Arelli 1994).

As mentioned previously, G. max_{[}p_{eg]} and G. max_{[PI 88788]} are the archetypal sources of almost all the germplasm that is bred into commercial varieties of soybean. The underlying nature of G. max_{[Peking]} resistance is rhg1, rhg2, and rhg3, accompanied by the dominant gene Rhg4 (Matson and Williams 1965). The G. max_{[PI 88788]} resistance is explained by rhg1, rhg2, Rhg4 and Rhg5 (Glover et al. 2004; reviewed in Concibido et al. 2004). Those studies document that both G. maxp_{ekingi} and G. max_{[PI 88788]} harbor rhg1, rhg2, and Rhg5 while G. max_{[}p_{eg]} has rhg3 and G. max_{[PI 88788]} has Rhg5. Other less well understood resistance factors have been identified through quantitative trait loci (QTL) mapping studies in G. maxpₑki_{ng}i and G. max_{[PI 88788]} (reviewed in Concibido et al. 2004).

Understanding the nature of the resistance genes is not a straightforward process. The complication comes from the number of field isolated populations of H. glycines that have varying capacities to infect the numerous G. max genotypes (Ross 1962). Currently, there are 16 historically accepted H. glycines races (Riggs and Schmitt 1988, 1991; Riggs 1988). However, more recent research has resulted in a reclassification of H. glycines races as distinct populations because they can only be maintained through a sexual reproductive cycle (Niblack et al. 2002). The reclassification scheme was made possible because more G. max genotypes that exhibit various levels of resistance are available and more sophisticated tests have been developed over the years that could tease out the fine details of the infective capability of unknown H. glycines populations (Niblack et al. 2002). The identity of an unknown field isolated population is determined by an H. glycines type (HG-type) test. During the HG-type test, the nematode is allowed to infect a known susceptible genotype along with a panel of seven or more G. max genotypes with varying abilities to resist infection by the different H. glycines populations (Niblack et al. 2002). The HG-type test is based off of other studies (Ross 1962; Golden et al. 1970; Riggs and Schmitt 1988, 1991; Riggs 1988). Thus, the revised HG-type test is an important development in H. glycines research. However, the HG-type test does not provide information on how various G. max genotypes accomplish resistance at the site of infection (i.e., the syncytium). Such information would provide useful knowledge in understanding how each resistant G. max genotype alters their gene expression to accomplish resistance.

The resistant reaction at the syncytium undergoes two distinct developmental phases that have been documented at the cytological and ultrastructural levels (Endo 1965, 1991; Riggs et al. 1973; Acido et al. 1984; Kim et al. 1987; Halbrendt et al. 1992; Kim and Riggs 1992; Mahalingham and Skorupska 1996; Klink et al. 2007a, b, 2009a, 2010a, b). The first phase (phase 1) occurs when the nematode appears to be engaging the parasitism machinery to initiate the formation of the syncytium. During phase 1, the syncytium of both resistant and susceptible reactions appears the same (Endo 1965; Riggs et al. 1973; Acido et al. 1984; Kim et al. 1987). Phase 1 includes the dissolution of cell walls, enlargement of nuclei, limited hypertrophy, the presence of dense cytoplasm and increased ER content (Endo 1965; Riggs et al. 1973; Kim et al. 1987). Phase 1 occurs between 1 and 4 days post inoculation (dpi), depending on the genotype of G. max (Endo 1965; Riggs et al. 1973; Kim et al. 1987). The second phase (phase 2) of the resistance reaction becomes evident at both the cytological and ultrastructural levels by 4-5 dpi (Endo 1965; Riggs et al. 1973; Acido et al. 1984; Kim et al. 1987). The resistance characteristics are dependent on the genotype of G. max.

A rudimentary classification scheme of G. max resistance has been developed from the cytological, ultrastructural and developmental comparative analyses of how the various G. max genotypes react to H. glycines (Colgrove and Niblack 2008). The work has resulted in the designation of the G. max_{[}p_{eking]} and G. max_{[PI 88788]} groups (Colgrove and Niblack 2008; FIG. 1). The designation of the G. max_{[}p_{eking]} and G. max_{[PI 88788]} groups are based on numerous observations (Ross 1958; Endo 1965; Riggs et al. 1973; Acido et al. 1984; Kim et al. 1987; Halbrendt et al. 1992; Kim and Riggs 1992; Mahalingham and Skorupska 1996). The G. max_{[}p_{eking]} group includes the genotypes G. max_{[}p_{eking]}, G. max_{[pi 90763]}, G. max_{[pi 89772]} and partially G. max_{[pi 437654]}. The G. max_{[PI 88788]} group includes G. max_{[PI 88788]}, G. max_{[PI 209332]} and G. max_{[PI 548316]} (Colgrove and Niblack 2008). In addition to documenting the cellular reaction of the syncytium as it degenerates as a consequence of its interaction with the nematode, the work has also documented the differences in stages when nematode development arrests during the G. max_{[Peking]} and G. max_{[PI 88788]}-types of resistant reactions.

Cytological work has documented that there is an earlier response to nematode infection during the G. max_{[}p_{eking]} type of resistant reaction (Endo 1965; Riggs et al. 1973; Acido et al. 1984; Kim et al. 1987; Kim and Riggs 1992). Cytological observations reveal that the G. max_{[}p_{eking]}-type of resistance response involves necrosis of the syncytial cells that surround the head of the nematode. The necrotic layer separates the syncytium from the surrounding cells (Kim et al. 1987). The resistant reaction in the G. max_{[}p_{eking]} archetype has a characteristic cytology that accompanies these cytological features. The G. max_{[}p_{eking]} resistant reaction includes the formation of cell wall appositions (CWA). The CWAs are structures defined as physical and chemical barriers to cell penetration (Aist 1976; Schmelzer 2002; Hardham et al. 2008). However, syncytia continue their later stages of the resistant reaction even at 7 dpi in G. max_{[}p_{eking]} (Riggs et al. 1973). The resistant reaction is accompanied by the degeneration of the p-J2 nematode within 4-5 dpi (Endo 1964, 1965; Kim et al. 1987; Kim and Riggs 1992). Consequently, the G. max_{[}p_{eking]}-type of resistance response blocks glycines development at the p-J2 stage (Endo 1965; Riggs et al. 1973).

In contrast, studies in a derivative of G. max_{[PI 88788]}, known as G. max[Bedfordtpi 548974] revealed a different cytological response (Hartwig and Epps 1978). The G. max_{[Bedford/PI 548974]} resistance reaction lacks the development of a necrotic layer by 5 dpi (Kim et al. 1987). This is an important distinction between the G. max_{[}p_{eking]} and G. max_{[PI 88788]} types of resistant reactions because a necrotic layer is formed in G. max_{[}p_{eking]}. The initial stages of the G. max_{[PI 88788]}-type of resistant reaction involves extensive accumulation of cisternae and rough ER and nuclear degeneration within the syncytium by 5 dpi (Kim et al. 1987). There are no thickened cell walls or CWAs that form. Degradation of the cytoplasm is observed by 10 dpi (Kim et al. 1987). The G. max_{[PI 88788]}-type of resistance reaction results in nematode death at the J3 and J4 stages (Acido et al. 1984; Kim et al. 1987; Colgrove and Niblack 2008) which is later than that observed for the G. max_{[}p_{eking]}-type of reaction.

The observations demonstrate that the G. max_{[}p_{eking]}-type of resistance reaction is potent and rapid while the G. max_{[PI 88788]}-type is potent but prolonged. The research shows the merit of categorizing resistance as the G. max_{[}p_{eking]}-type or the G. max_{[PI 88788]}-type. This is because it places the reactions into a useful context at the cellular and developmental levels for both the plant and the parasite. By knowing the chronology of the reactions, the time points can be used as benchmarks for the design of gene expression experiments that can test what underlies the different forms of the resistant reactions. To perform these studies, reliable methods are required to isolate the nurse cells undergoing the resistant reactions.

Important molecular information on the details of the resistant reactions of plants to parasitic nematodes has been obtained by isolating syncytia by laser capture microdissection (LCM) (Klink et al. 2005, 2007b, 2009c, 2010a, b). The LCM procedure is a proven method to faithfully isolate homogeneous cell populations from complex tissue (Isenberg et al. 1976; Meier-Ruge et al. 1976; Emmert-Buck et al. 1996; Asano et al. 2002). The LCM-based studies investigating the resistant reaction occurring at the site of infection were accompanied by molecular and computational investigations (Klink et al. 2005, 2007a, 2009a, 2010a, b). RNA isolated from syncytia undergoing resistant or susceptible reactions has been used to make cDNA libraries and clone full length genes (Klink et al. 2005). The cDNAs synthesized from microdissected syncytia has been used for making probes for RNA in situ hybridization and to perform quantitative measures of gene expression (qRT-PCR) (Klink et al. 2005). RNA isolated from syncytia has also been used to compare resistant and susceptible reactions in microarray analyses (Klink et al. 2007a, 2009a, 2010a, b). Some of these studies have relied on multiple genetically distinct H. glycines populations to obtain resistant or susceptible reactions in a single G. max genotype (Klink et al. 2007a, 2009a, 2010b). Such experiments provide an unambiguous comparative analysis of resistant and susceptible reactions because no genetic differences exist in the plant genotype.

The analyses identified many induced and suppressed genes and gene pathways in G. max[Peking/PI 548402] and G. max_{[PI 88788]} as compared to their respective genotype-specific pericycle and surrounding cell populations (Klink et al. 2007a, 2009a, 2010a). Prior analyses identified differential expression in the form of induced gene expression where expression is measurably higher in the syncytium than a control population of cells during a resistant reaction (Klink et al. 2007a, 2009a, 2010a). The analyses also identified differential expression in the form of suppressed gene activity where expression is measurably lower than a control population of cells during a resistant reaction (Klink et al. 2007a, 2009a, 2010a). The analyses have identified induced levels of genes involved in lipoxygenase signaling and phenylpropanoid metabolism among others (Klink et al. 2007a, 2009a, 2010a). While these studies have revealed some of the intricacies of the individual resistant reactions occurring in the G. max_{[Peking/PI 548402]} and G. max_{[PI 88788]} genotypes, they did not provide a comparative analysis of the different forms of the resistant reaction.

Since comparative studies between the different forms of the resistant reaction have not been done, analyses of modulations in gene expression that relate to the different forms of the resistant reaction could not be addressed. Modulation is defined as changes in gene activity that are based on the genotype of the plant, in the case presented here, the form of the resistant reaction. Modulation is a property that is different than differential expression. Modulation is different because in modulation, the activity state of the gene pertains to a specific point of time during a developmental process in comparisons between different genotypes. Thus, a gene can experience differential expression (i.e., an induced state) as compared to a control cell population while also being amplified in its expression as compared to a different G. max genotype (i.e., G. max_{[Peking/PI 548402]} VS. G. max_{[PI 88788]}). Alternatively, the modulated gene activity can be attenuated. Attenuation is defined as the activity of a gene being lower in one genotype as compared to the other. Thus, a gene can be experiencing induced activity and also be attenuated in comparisons between different genotypes at a specific time point.

### SUMMARY OF THE INVENTION

Glycine max L. Merr. (soybean) resistance to Heterodera glycines Ichinohe occurs at the site of infection, a nurse cell known as the syncytium. Resistance is classified into two cytologically-defined responses, the G. max_{[}p_{eking]}- and G. max_{[PI 88788]-types. Each type represents a cohort of} G. max genotypes. Resistance in G. max_{[}p_{eking]} occurs by a potent and rapid localized response, affecting parasitic second stage juveniles (p-J2). In contrast, resistance occurs by a potent but more prolonged reaction in the genotype G. max_{[PI 88788]} that affects nematode development at the J3 and J4 stages.

Microarray analyses comparing these cytologically and developmentally distinct resistant reactions reveal differences in gene expression in pericycle and surrounding cells even before infection. The differences include higher relative levels of the differentially expressed in response to arachidonic acid 1 gene (DEA1 [Gm-DEA1]) (+224.19-fold) and a protease inhibitor (+68.28-fold) in G. max_{[Peking/PI 548402]} as compared to G. max_{[PI 88788]}.

Gene pathway analyses compare the two genotypes (1) before, (2) at various times during, (3) constitutively throughout the resistant reaction and (4) at all time points prior to and during the resistant reaction. The amplified levels of transcriptional activity of defense genes may explain the rapid and potent reaction in (G. max_{[Peking/PI 548402]}) as compared to G. max_{[PI 88788]}. In contrast, the shared differential expression levels of genes in G. max_{[Peking/PI 548402]} and G. max_{[PI 88788]} may indicate a conserved genomic program underlying the G. max resistance on which the genotype-specific gene expression programs are built off.

### BRIEF DESCRIPTION OF THE DRAWINGS

Further advantages of the invention will become apparent by reference to the detailed description of preferred embodiments when considered in conjunction with the drawings:

FIG. 1 depicts G. max_{[Peking/PI 548402]} and G. max_{[PI 88788]} resistant reactions.

FIG. 2 depicts Histological responses of G. max_{[Peking/PI 548402]} and G. max_{[PI 88788]} roots to H. glycines infection during their resistant reactions.

FIG. 3 depicts a microdissected syncytium.

FIG. 4 depicts volcano plots depicting relative gene expression.

FIG. 5 depicts a Pathway analysis and comparison of the brassinosteroid biosynthesis pathway.

FIG. 6 shows volcano plots comparing differential gene expression of the 3, 6 or 9 dpi_{[Peking/PI 548402+PI 88788]} combined syncytium samples to the pericycle_{[Peking/PI 548402+PI 88788}] combined sample.

FIG. 7 depicts line graph depicting genes that are induced or suppressed in syncytium samples at the 3, 6 and 9 dpi time points as compared to pericycle samples in both G. max_{[Peking/PI 548402]} and G. max_{[PI 88788]}.

### DETAILED DESCRIPTION

The following detailed description is presented to enable any person skilled in the art to make and use the invention. For purposes of explanation, specific details are set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

Research on the genetic program underlying how a single genetically pure, inbred population of H. glycines elicits the development of two very different forms of the resistant reaction at the site of infection would determine commonalities and unique features of the different reactions. This is an important point because the genetic program accompanying the contrasting resistant reactions involves a dramatic change in the rate of the reaction that is dependent on the genotype of plant that is infected. Comparative analyses of the different forms of the resistant reaction can be done by using G. max_{[Peking/PI 548402]} and G. max_{[PI 88788]} because they have been extensively studied at the cytological level.

We disclose herein a method for the identification of the nematode resistance genes in important agricultural plants such as G. max. The microarray analysis presented here compares the resistance processes of G. max_{[Peking/PI 548402]} and G. max_{[PI 88788]}. This d represents a unique perspective on how gene expression is occurring during the resistant reaction in two soybean genotypes that both share while also having unique sets of resistance genes. Importantly, a single H. glycines race, H. glycines_{[NLi}_{_{—}}_{migi}, determined to be HG-type 7 (race 3) (Klink et al. 2007a, 2009a, 2010a) is used to obtain the diverse resistant reaction types observed in G. max_{[Peking/PI 548402]} and G. max_{[PI 88788]}. The time points selected for the analysis span both the parasitism and resistance phases of the resistant reaction (Klink et al. 2009a, 2010a).

The work moves beyond prior investigations by showing genes and gene pathways that experience differential expression and modulated gene activity. This was accomplished because the analyses investigated the rapid and potent resistant reaction of G. max_{[Peking/PI 548402]} as compared to the prolonged but potent resistant reaction of G. max_{[PI 88788]}. These observations answer the question of whether modulated gene expression underlies the G. max_{[Peking/PI 548402]} and G. max_{[PI 88788]} forms of the resistant reaction. The work also identifies differential gene expression that is common between the G. max_{[Peking/PI 548402]} and G. max_{[PI 88788}] genotypes. This work in its entirety shows that all localized resistant reactions at the syncytium of the different G. max genotypes are not under the same genetic control and/or involve the same genomic imprint. The work also shows that a common gene expression pattern is present between G. max_{[Peking/PI 548402]} and G. max_{[PI 88788]} that may represent a generalized physiological platform in action that underlies a broad spectrum resistance to H. glycines.

Materials and Methods Experimental Methods

The materials and methods pertaining to H. glycines populations, G. max genotypes, experimental procedures and data analyses are published in Klink et al. 2009a and Klink et al. 2010a, herein incorporated by reference.

Plant and Nematode Procurement

Plant and nematode procurement methods are published (Klink et al. 2005, 2007a, b, 2009a, b, c, 2010a, b; Alkharouf et al. 2006). The plant introduction (PI) identifier G. max_{[}p_{eking]} genotype used in the analyses is PI 548402 (G. max_{[Peking/PI 548402]}). The PI identifier for G. max[Peking] is important because it has seven different plant introductions (PI 297543, P1438496 A, P1438496 B, PI 438496 C, PI 438497, PI 548402S) of unclear association. Unlike G. max_{[}p_{eking]}, G. max_{[Peking/PI 88788]} is unique. The G. max_{[Peking/PI 548402]} and G. max_{[Peking/PI 88788}] stocks were originally obtained from a seed bank that is managed and maintained by the United States Department of Agriculture (USDA). Seeds were made available through the National Plant Germplasm System (http://www.ars-grin.gov/npgs/acc/acc_queries.html). The H. glycines NL1-RHg population used in the studies is race 3, HG-type 7 (H. glycines_{[NL1-RHg/HG-type 7])} as determined in the independent lab of Dr. Terry Niblack (Department of Crop Sciences, University of Illinois) June-July 2007 using the methods of Niblack et al. (2002) (Klink et al. 2009a, 2010a).

The HG-type test also determined that G. max_{[Peking/PI 548402]} and G. max_{[PI 88788]} are considered highly resistant to (H. glycines_{[NL1-RHg/HG-type 7])} (Klink et al. 2009a, 2010a). Consistency of the performance of G. max_{[Peking/PI 548402]} and G. max_{[PI 88788]} genotypes are tested and confirmed for each experiment (Klink et al. 2009a, 2010a). The G. max_{[Peking/PI 548402]} and G. max_{[PI 88788]} genotypes are used in the experiments to obtain resistant reactions by the use of (H. glycines_{[NL1-RHg/HG-type 7])}. The (H. glycines_{[NL1-RHg/HG-type 7])} population is maintained in the greenhouse using the moisture replacement system (Sardanelli and Kenworthy 1997). Prior to infection, the nematodes are diluted to a final concentration of 2,000 pi-J2/ml. This quantity of nematodes is added to each root of each plant. The roots, including the mockinfected control samples, are washed after 1 day to remove nematodes that had not penetrated the roots. Infected roots are grown for 3, 6 or 9 dpi. Maximally infected lateral roots are harvested for analyses. The process is subsequently repeated twice, providing three independent sets of samples.

Histology

Histological observation is performed according to Klink et al. (2005, 2007a, b, 2009a, b, c, 2010a, b). Briefly, tissue is fixed in Farmer's solution (FS) composed of 75% ethanol, 25% acetic acid (Sass 1958; Klink et al. 2005). Tissue is cast and subsequently mounted for sectioning. Serial sections of roots are made on an American Optical 820® microtome (American Optical Co®.; Buffalo, N.Y., USA) at a section thickness of 10 gm. Sections are stained in a manner similar to the original experiments of Ross (1958) and Endo (1965). Staining involves Safranin 0 (Fisher Scientific Co.; Fair Lawn, N.J., USA) in 50% ETOH and counter-staining in Fast Green FCF (Fisher Scientific Co.) (Klink et al. 2005). For histological analyses, the tissue is permanently mounted in Permount® (Fisher Scientific Co.).

LCM and Microarray Hybridization

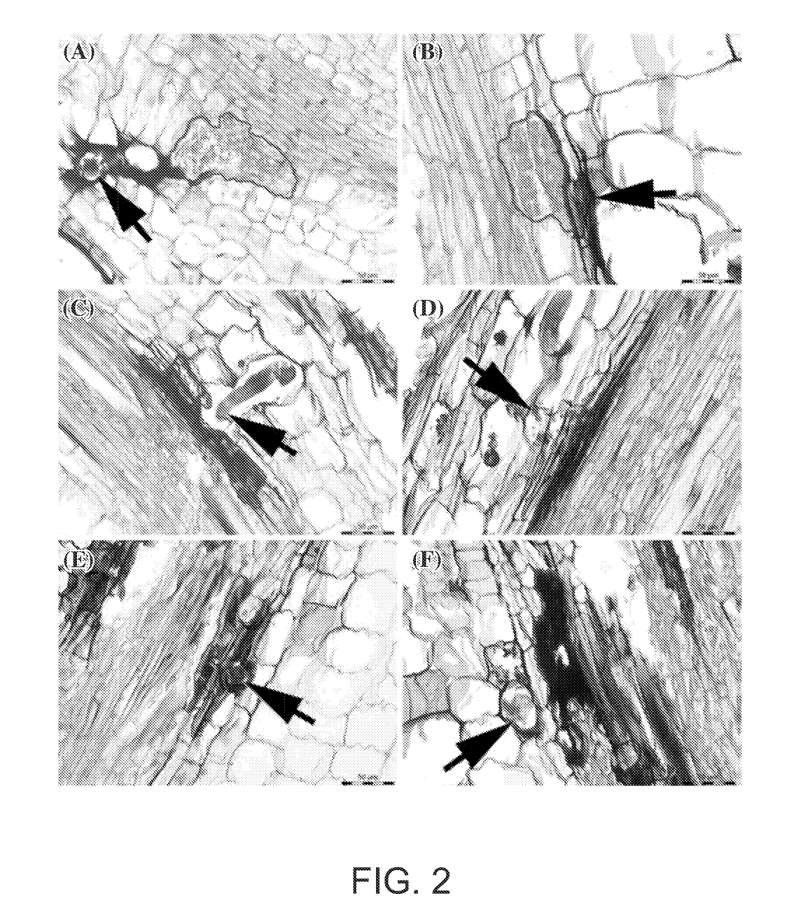

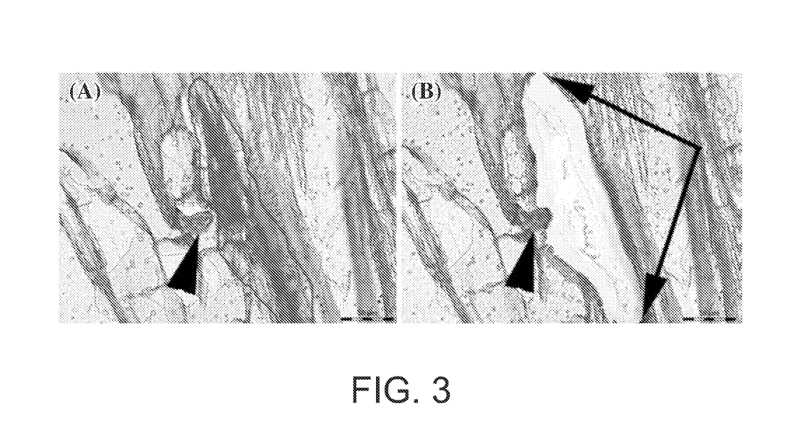

Slides are prepared according to Klink et al. (2005, 2007a, 2009a, 2010a, b). LCM is performed on a Leica® ASLMD Microscope® (Leice). Syncytia are simple to visualize (FIG. 2) and microdissect (FIG. 3). What is difficult to do is estimate the actual number of cells used in the studies. This is because serial sections of paraffin-embedded tissue were used for LCM of the control and syncytium cells. In addition, by definition, the syncytium is composed of multiple fused cells that are difficult to differentiate from each other. Also, the cells under study were undergoing a resistant reaction, complicating visualization of the individual cells that compose the syncytium. Serial sections of approximately 100 syncytia were used to obtain the RNA for the studies for each replicate. Seven sub-replicates were made for each replicate. Over 100 ng of RNA per replicate was obtained for the studies. Work to obtain RNA is done with the PicoPure RNA Isolation kit (Molecular Devices® [formerly Arcturus®]; Sunnyvale, Calif., USA, Cat. # KIT0204). A DNAse treatment is added, just before the second column wash, using DNAfree® (Ambion®, Austin, Tex., USA). RNA quality and yield are determined using the RNA 6000 Pico Assay® (Agilent Technologies®, Palo Alto, Calif., USA) using the Agilent 2100 Bioanalyzer® according to the manufacturer's instructions. Both probe preparation and hybridization procedures on the GeneChip® Soybean Genome Array (Affymetiix®, Cat. #900526) was performed according to Affymetrix® guidelines.

Data Analysis

The soybean GeneChip® data is imported and analyzed using the MATLAB Bioinformatics Toolbox (Mathworks Inc.; Natick, Mass., USA). Log 2 scaling is used to standardize the dataset. Volcano plots are produced using samples having a fold change of >I±1.51 and also having a P value <0.05 as compared to the control (Alkharouf et al. 2006) and false discovery rate (FUR) less than 10% (Klink et al. 2009a, 2010a). Annotations are made by comparison to the Arabidopsis thaliana gene ontology (GO) database (The Gene Ontology Consortium 2004) based on their best match obtained by BLAST searches (Altschul et al. 1997). Annotations were subsequently re-run in order to identify additional best-hit matches. Annotations were updated September 2010.

Gene Pathway Analyses

The pathway analysis visualizes pathways according to Kyoto Encyclopedia of Genes and Genomes (KEGG) (http://www.genome.jp/kegg/catalog/orglist.html) from Affymetrix® gene expression data. In the pathway analysis, the darker the shade of green represents the greater the level of induced gene expression as compared to controls or amplified expression as compared to the other genotype. Yellow represents expressed. The darker the shade of red means the greater the suppressed level gene expression or lower expression as compared to the other genotype. Data supplemental to each table and figure and GO terms (The Gene Ontology Consortium 2004) are available (http://bioinformatics.towson.edu//).

Results

TABLE 1
A comparative analysis of G. max[Pcking/PI 548402] pericycle to G. max[PI 88788]
relative levels of expression

| | | | | | |
|---|---|---|---|---|---|
| Probe ID | GenBank ID | Description | PC | PV | q value (%) |

Peking pericycle-induced

| | | | | | |
|---|---|---|---|---|---|
| GmaAffx.80464.1.SLat | CA850542 | Arachidonic acid-induced DEA1 | 224.1921 | 4.14E−05 | 0 |
| Gma.677.I.SLat | BU082252 | Protease inhibitor | 68.27514 | 7.46E−04 | 0 |
| GmaAffx.92741.1.S1 s at | CF808466 | Polygalacturonase-like protein | 66.07655 | 7.17E−03 | 0 |
| Gma.1043.I.SLat | B1785070 | R-gene | 55.06712 | 5.68E−05 | 0 |
| Gma.17364.I.SLat | CA803078 | P24 oleosin isoform A | 53.78355 | 2.46E−04 | 0 |
| Gma.7993.I.SLat | BI967267 | Extensin-like protein | 49.69328 | 1.38E−05 | 0 |
| Gma.5315.I.S Lat | CD409280 | Lipoxygenase, LH2 | 44.88481 | 2.14E−04 | 0 |
| GmaAffx.1663 2.I.S1 at | AW598371 | NA | 39.42695 | 8.56E−03 | 0.4497689 |
| GmaAffx.71067.I.SLat | AW307334 | NA | 38.62491 | 1.66E−02 | 0.2361532 |
| Gma.3308.1.S Lat | CD414773 | Polygalacturonase-like protein | 37.9182 | 5.46E−05 | 0 |
| GmaAffx.11899.I.ALat | AW759403 | Glycosyl hydrolase family 1 protein | 35.06023 | 7.18E−05 | 0 |
| Gma.16558.I.SLat | CD401715 | ATSUFE/CPSUFEIEMB1374 (EMBRYO | 31.79591 | 8.06E−03 | 0 |
| | | DEFECTIVE 1374) | | | |
| GmaAffx.88165.1.SLat | BE583573 | STP1 (SUGAR TRANSPORTER 1) | 31.15182 | 1.19E−02 | 0 |
| GmaAffx.27712.1.SLat | AW707016 | NA | 29.18668 | 1.39E−05 | 0 |
| GmaAffx.73486.I.S1 s at | BM092012 | Lipoxygenase, LH2 | 28.98119 | 5.81E−03 | 0 |
| Gma.15398.I.SLat | BG651396 | Zwille protein-like | 27.62509 | 1.52E−03 | 0 |
| Gma.8494.I.S Lat | AF128266 | Polygalacturonase PG1 | 27.43525 | 1.05E−02 | 0.3437161 |
| Gma.6146.1.SLat | BI969869 | Putative 14-kDa proline-rich protein | 27.41625 | 2.21E−03 | 0 |
| Gma.3591.1.Sl a at | BE440266 | ACC oxidase | 25.51494 | 2.80E−02 | 0.8628225 |
| GmaAffx.75044.1.A1 at | CD397448 | NA | 24.84929 | 5.02E−04 | 0 |
| GmaAffx.69913.1.ALat | BU550803 | NA | 24.21203 | 1.86E−03 | 0 |
| Gma.2505.1.SLat | AB062754 | Ferritin-2, chloroplast precursor | 21.16031 | 1.50E−02 | 0 |
| Gma.684.2.ALat | BU084534 | Cold acclimation WCOR413 | 21.04413 | 2.67E−02 | 0.3437161 |
| Gma.8299.1.S Lat | BI967830 | Protein phosphatase-2c | 20.57819 | 4.42E−03 | 0.2361532 |
| Gma.772.I.SLat | BE819852 | Hypothetical protein | 20.57294 | 2.66E−04 | 0 |
| GmaAffx.36115.I.ALat | BE659438 | NA | 20.4504 | 1.05E−03 | 0 |
| GmaAffx.80486.1.S Lat | AW733824 | Pectin acetylesterase | 20.17877 | 8.96E−03 | 0.2361532 |
| GmaAffx.49549.1.SLat | BG363167 | Synaptobrevin-related | 20.16506 | 1.14E−03 | 0 |
| GmaAffx.65885.1.ALat | CD405808 | Zinc finger (C2H2 type) | 20.12014 | 4.78E−03 | 0.2361532 |
| GmaAffx.9605.1.SLat | BM091603 | NA | 20.09384 | 6.03E−04 | 0 |

FC Fold change,
PV P value.
A C|±1.5| fold cutoff and P B 0.05 was used. FDR was set to 12%. The probe set satisfied the criteria set by the differential expression and FDR analyses.

TABLE 2
A comparative time course analysis of G. max[Pcking/PI548402] 3, 6 and 9 dpi to G. max[PI88788](baseline) 3, 6 and 9 dpi resistant reaction

| | GenBank | | Control | 3 day | 6 day | 9 day |
|---|---|---|---|---|---|---|
| Probe ID | ID | Description | FC | FC | FC | FC |
| HIGHER in G. maxl Peking] | | | | | | |
| GmaAffx.91687.1.Als at | CF807412 | Protein disulfide isomerase (pD I)-like protein 2 | NA | 16.42058 | 22.66097 | 16.55722 |
| Gma.6245.1.S Lat | CA938361 | Thaumatin | NA | 15.98745 | 21.85067 | 11.51146 |
| Gma.2044.1.S Lat | M94012 | RESPONSIVE TO ABA 18 (RABI8) | NA | 12.63209 | 19.24757 | 24.24861 |
| GmaAffx.80465.1.S 1 at | BU548370 | ARABIDOPSIS THALIANA GALACTINOL | NA | 11.84203 | 7.204501 | 11.41504 |
| | | SYNTHASE 2 (ATGOLS2) | | | | |
| Gma.11135.1.SLat | A1856724 | ATP-citrate lyase A-3 (ACLA-3) | NA | 11.60219 | 3.023322 | 6.155099 |
| Gma.3988.1.S l--at | BE658819 | Lactoylglutathione lyase family protein | NA | 9.953863 | 10.4648 | 9.34481 |
| Gma.3473.1.S Lat | BQ628660 | 17.6 kDa class I small heat shock protein | NA | 9.283021 | 4.034156 | 2.918122 |
| | | (HSPI7.6B-CI) | | | | |
| Gma.391.1.ALat | AW349964 | Receptor-like protein kinase-related | NA | 8.295254 | 3.48455 | 7.635437 |
| GmaAffx.60625.1.S 1 at | BQ740664 | NA | NA | 7.422014 | 4.368441 | 4.198288 |
| Gma.2912.1.S Lat | AF117884 | Late embryogenesis abundant protein (I FA) | NA | 7.174836 | 6.243753 | 5.686975 |
| Gma.173.1.SLat | X63565 | Late embryogenesis abundant group 1 (LEAL) | NA | 6.270906 | 18.14174 | 6.578355 |
| Gma.1950.1.SLat | AE202184 | Isoflavone reductase | NA | 5.996244 | 2.927154 | 8.598727 |
| Gma.2912.1.S Ls_at | AF117884 | Late embryogenesis abundant protein (LEA) | NA | 5.989421 | 12.04405 | 10.04885 |
| Gma.4616.1.ALat | AW349487 | Thaumatin | NA | 5.880116 | 5.885979 | 6.056813 |
| GmaAffx.91687.1.S 1...at | CF807412 | Protein disulfide isomerase (PD I)-like protein 2 | NA | 5.820802 | 5.591163 | 4.333533 |
| GmaAffx.15955.1.SLat | AI495284 | CPC (CAPRICE) transcription factor | NA | 5.625248 | 5.174757 | 5.123017 |
| Gma.12393.1.SLa..at | BM093715 | NINE-CIS-EPOXYCAROTENOID | NA | 5.587755 | 4.731004 | 3.145295 |
| | | DIOXYGENASE3 (NCED3) | | | | |
| Gma.5637 .1.S Lat | AY126715 | ARABIDOPSIS THALIANA GALACTINOL | NA | 5.496911 | 7.509643 | 5.386323 |
| | | SYNTHASE 1 (ATGOLS1) | | | | |
| LOWER in G. maxl Peking] | | | | | | |
| Gma.5949.1.ALat | CD412339 | Histone H3 | NA | 48.089 | −9.2502 | −6.0738 |
| GmaAffx.82904.1.SLat | BM885134 | NA | NA | −22.245 | −9.11273 | −8.6476 |
| Gma.l0969.3.SLx_at | CD396552 | Lipoxygenase | NA | −16.288 | −16.2558 | −4.7436 |
| Gma.11166.1.SLx_at | AW705829 | Lipoxygenase 1 (LOX1) | NA | −12.04 | −13.7154 | −7.1331 |
| Gma.6037 .1.S Lat | CD390794 | Hydroxycinnamoyl transferase | NA | −10.677 | −26.6953 | −15.725 |
| Gma.7890.1.ALat | AW309342 | Pectinesterase family protein | NA | −8.6387 | −6.53887 | −4.8652 |
| Gma.13208.2.ALat | CD392188 | ZPR 2 LITTLE ZIPPER 2 | NA | −8.4889 | −8.11234 | −6.366 |
| GmaAffx.54898.1.S 1 s at | BQ452830 | Extensin | NA | −5.3104 | −3.03486 | −4.4452 |
| Gma.3905.1.S Lat | BU547451 | NA | NA | −4.6281 | −2.51223 | −6.4595 |
| Gma.8520.1.S Lat | CA802722 | Acid phosphatase, putative | NA | −4.628 | −10.516 | −12.966 |
| GmaAffx.4050.1.S1 at | CD393498 | Microtubule motor | NA | −3.7378 | −5.2331 | −4.4288 |
| GmaAffx.73185.1.S Lat | B1701903 | NA | NA | −3.0654 | −7.76978 | −6.1235 |
| Gma.15577.1.SLat | CD399075 | NA | NA | −2.3505 | −3.42822 | −5.0828 |
| GmaAffx.24622.1.S Lat | BU926989 | NA | NA | −2.2107 | −2.38433 | −5.154 |

Fe Fold change, PV P value. The analysis shows genes with higher relative levels of expression. A :::| ± 1.51 fold cutoff and P ::: 0.05 was used.
FDR was set to 12%. The probe set satisfied the criteria set by the differential expression and FDR analyses.
Full information for the 3-point time course analysis (Supplemental Table 5) is provided

TABLE 3
A comparative time course analysis of G. max[Pcking/PI548402]pericycle, 3, 6
and 9 dpi to G. max[pI88788] (baseline) pericycle, 3, 6 and 9 dpi resistant reaction

| | GenBank | | Control | 3 day | 6 day | 9 day |
|---|---|---|---|---|---|---|
| Probe ID | ID | Description | FC | FC | FC | FC |
| HIGHER in G. Maxl Peking] | | | | | | |
| Gma.16558.1.SLat | CD401715 | EMBRYO DEFECTIVE 1374 | 31.79591 | 21.20895 | 26.4705 | 26.49603 |
| | | (EMBI374) transcription regulator | | | | |
| GmaAffx.69913.1.Al -- at | BU550803 | NA | 24.21202 | 5.051458 | 2.916091 | 6.453139 |
| Gma.3440.1.S 1 s at | BE820653 | Annexin | 14.12382 | 54.12197 | 92.66703 | 13.48165 |
| Gma.2527 .1.S Ls_at | AW310149 | Hydrolase | 11.08717 | 5.22427 | 2.996417 | 5.736873 |
| Gma.8075.1.SLat | BG237495 | ARABIDOPSIS THALIANA | 10.19415 | 5.114079 | 5.158075 | 3.881952 |
| | | ROOT FNR 2 oxidoreductase | | | | |
| | | (ATRFNR2) | | | | |
| Gma.5213.1.SLat | BI970096 | THIAZOLE REQUIRING (THI1) | 10.15316 | 8.158338 | 6.591349 | 5.271578 |
| Gma.2102.1.S1 s at | BM523392 | Disease resistance-responsive | 9.850742 | 8.057176 | 7.452264 | 14.15474 |
| | | family protein/fibroin-related | | | | |
| GmaAffx.51673.1.S 1 at | BI892643 | Short-chain dehydrogenase/ | 9.722524 | 2.64891 | 3.870993 | 3.53163 |
| | | reductase (SDR) family protein | | | | |
| Gma.17814.1.SLat | AW349375 | SMALL AND BASIC INTRINSIC | 8.256599 | 4.366413 | 2.854187 | 8.04231 |
| | | PROTEIN 1A (SIPl; 1) | | | | |
| Gma.4675.1.ALat | AW309784 | NA | 7.710598 | 4.218979 | 2.518369 | 3.656552 |
| Gma.6742.1.ALat | BU544682 | NA | 5.91864 | 3.658485 | 1.933151 | 6.97982 |
| GmaAffx.65225.1.Al at | BU544196 | NA | 5.310171 | 2.118906 | 2.690097 | 2.07855 |
| Gma.9206.1.ALat | BE658223 | DNAJ/HSP40 homolog | 5.133189 | 65.02919 | 52.75578 | 24.94265 |
| GmaAffx.92230.1.Als at | CF807955 | ARABIDOPSIS THALIANA | 5.120276 | 5.125551 | 1.916674 | 8.772659 |
| | | OSMOTIN 34 (ATOSM34) | | | | |
| Gma.6523.1.S Lat | CD416204 | COI1 SUPPRESSORI (COS1) | 4.788784 | 2.881405 | 2.25572 | 7.180979 |
| | | 6,7-dimethyl-8-ribityllumazine | | | | |
| | | synthase | | | | |
| Gma.2048.1.S Lat | BQ273518 | Nucleolar protein gar2-related | 4.777935 | 2.453995 | 1.605651 | 2.10036 |
| GmaAffx.91617.1.S Lat | CF807342 | NA | 4.367093 | 2.467069 | 3.213129 | 2.361197 |
| Gma.12929.1.ALat | BU546100 | Aspartyl protease family protein | 2.818529 | 3.993042 | 1.854135 | 3.546448 |
| Gma.16111.2.ALat | BU550402 | ACTIN 3 (ACT3) | 2.79373 | 2.501857 | 2.866219 | 4.494345 |
| Gma.7795.1.SLat | AW348257 | Rubber elongation factor (REF) | 2.729386 | 3.07138 | 1.864679 | 2.939769 |
| | | family protein | | | | |
| Gma.1751.1.S l--at | BE473710 | Metalloendopeptidase (MPPBET A) | 2.628901 | 2.164241 | 1.962112 | 1.542794 |
| GmaAffx. 15031.I.S 1 at | B1702292 | RESPONSIVE TO DEHYDRATION | 2.376732 | 4.544688 | 2.310617 | 5.041949 |
| | | 19 (RDI9) cysteine-type peptidase | | | | |
| Gma.3483.1.S Lat | BQ628029 | Kelch repeat-containing F-box | 2.14176 | 4.415777 | 2.121393 | 4.804158 |
| | | family protein | | | | |
| Gma.2161.1.S La.at | CD404355 | U6 snRNA-associated Sm-like | 2.136185 | 1.547407 | 1.547049 | 1.890467 |
| | | protein | | | | |
| Gma.6474.1.ALx_at | B1788042 | ACCELERATED CELL DEATH 11 | 1.899929 | 2.102296 | 1.574223 | 1.937353 |
| | | (ACDll) | | | | |
| LOWER in G. MaxjPeking] | | | | | | |
| GmaAffx.90343.1.S Ls..at | CF806068 | GAST! PROTEIN HOMOLOG 4 | −2.302606 | −5.027036 | −6.657755 | −3.610013 |
| | | (GASA4) | | | | |
| Gma.2826.1.S Lat | CD397710 | TRANSPARENT TESTA 5 (TT5) | −5.625007 | −10.76999 | −14.78268 | −12.8567 |
| | | chalcone isomerase | | | | |
| GmaAffx.68100.1.S 1 at | BG508944 | Methionine synthase | −6.545762 | −11.58697 | −22.02279 | −18.49992 |
| GmaAffx.69560.1.S Ls.at | BQ628396 | Chloroplast 30S ribosomal | −14.02727 | −4.018961 | −12.89894 | −13.99015 |
| | | protein S3 | | | | |
| GmaAffx.38387.I.S Ls..at | BM525407 | NA | −4.462678 | −4.462678 | −13.47634 | −6.688526 |

Genes with higher and lower relative levels of gene expression are provided. Fe Fold change, PV P value. The analysis shows suppressed genes.
A :::1 ± 1.51 fold cutoff and P ::: 0.05 was used. FDR was set to 12%. The probe set satisfied the criteria set by the differential expression and FDR analyses.
Full information for the 4-point time course analysis Supplemental Table 6) is provided

TABLE 4

| | | | | | |
|---|---|---|---|---|---|
| Probe ID | GenBank ID | Description | 3 day FC | 6 day FC | 9 day FC |

Combined analysis-induced gene

| | | | | | |
|---|---|---|---|---|---|
| Gma.4837.1.S1_at | BI969343 | Nitrate transporter (NTP2) | 181.3572 | 145.4725 | 175.9298 |
| GmaAffx.92537.1.S1_at | CF808262 | Thioredoxin domain 2 | 175.9624 | 70.10148 | 179.7424 |
| GmaAffx.1301.90.S1_s_at | BU764871 | CPRD49 protein | 161.4081 | 146.0749 | 149.7704 |
| Gma.17258.1.Sl_s_at | BG509247 | Plasma membrane intrinsic polypeptide | 152.3878 | 82.58841 | 151.862 |
| Gma.17884.1.S1_s_at | BE023599 | Thioredoxin domain 2 | 99.4765 | 71.36182 | 97.50129 |
| GmaAffx.33851.1.S1_s_at | BG652918 | Plasma membrane intrinsic polypeptide | 88.00568 | 39.70022 | 81.5681 |
| Gma.3979.1.A1_at | BQ453202 | Oxidoreductase | 87.12431 | 75.71694 | 62.45248 |
| Gma.16908.1.S1_at | CA851713 | PLEIOTROPIC DRUG RESISTANCE 7 | 75.18718 | 73.5342 | 96.55285 |
| | | (ATPDR7/PDR7) | | | |
| GmaAffx.80899.1.S1_at | AW348263 | N/A | 53.88037 | 42.11188 | 70.05612 |
| Gma.8957.1.A1_at | BU764905 | Major latex protein-related | 47.88715 | 15.74441 | 11.18471 |
| Gma.4359.2.S1_at | BI968877 | Thioredoxin domain 2 | 45.34362 | 19.66089 | 37.8791 |
| Gma.5584.4.S1_s_at | BQ610991 | N/A | 45.278 | 88.56125 | 99.81554 |
| Gma.13252.1.S1_at | AW423744 | N0D26-LIKE INTRINSIC PROTEIN 4;1 | 43.53536 | 27.86329 | 41.55307 |
| Gma.15653.1.S1_at | B1969594 | CPRD49 protein | 40.63952 | 33.25427 | 39.99826 |
| GmaAffx.73751.1.S1_at | BM522529 | N/A | 38.84616 | 14.81812 | 23.665 |
| GmaAffx.89473.2.S1_s_at | CK605927 | N/A | 38.46777 | 84.22387 | 97.94334 |
| Gma.2091.1.S1_at | AW310549 | XYLOGLUCAN | 38.24392 | 25.02714 | 29.43529 |
| | | (XTR6) | | | |
| Gma.2133.1.S1_at | AW310071 | Alcohol acyl-transferases | 36.5949 | 17.34826 | 22.66635 |
| GmaAffx.74899.1.S1_at | CA852377 | N/A | 36.47777 | 35.29959 | 67.62322 |
| Gma.8365.1.S1_at | CD396813 | ALUMINUM SENSITIVE 3 (ALS3) | 35.51154 | 42.02656 | 38.02428 |
| GmaAffx.89473.2.A1_x_at | CK605927 | N/A | 33.28526 | 75.07749 | 94.55186 |
| GmaAffx.92561.1.S1_s_at | CF808286 | N/A | 30.65457 | 15.39094 | 20.09656 |
| Gma.5935.2.S1_a_at | AW278477 | N/A | 30.00026 | 16.70724 | 17.58698 |
| Gma.1756.1.S1_at | BF324935 | Vacuolar processing enzyme gamma | 29.66051 | 17.17768 | 10.50706 |
| | | (GAMMA-VPE) | | | |
| Gma.10827.3.S1_at | AW758966 | Oxidoreductase | 29.51436 | 18.3841 | 20.67453 |
| Gma.5935.1.S1_at | CA852006 | N/A | 29.35104 | 14.84596 | 16.74258 |
| GmaAffx.80180.1.A1_at | AW349390 | EPSP synthase | 29.23712 | 16.35677 | 23.59011 |
| Gma.10854.1.S1_at | CA802334 | Nodulin-like protein | 26.06708 | 25.32662 | 32.33187 |
| Gma.8130.1.S1_at | AI966352 | Malate synthase | 25.21859 | 27.49575 | 27.0197 |
| Gma.2771.1.S1_at | AW309763 | N/A | 24.77934 | 11.3021 | 12.5791 |
| Gma.5507.1.A1_s_at | BM108115 | N/A | 24.54817 | 22.31649 | 29.17702 |
| Gma.3668.1.S1_a_at | AW349263 | 1-Aminocyclopropane-1-carboxylate | 24.41935 | 29.84423 | 36.97905 |
| | | (ACC oxidase) | | | |
| Gma.3987.1.S1_at | BQ453408 | IMP dehydrogenase/GMP reductase | 24.37939 | 11.45596 | 13.33563 |
| GmaAffx.74127.1.S1_at | AW568912 | PLEIOTROPIC DRUG RESISTANCE 6 | 24.18316 | 29.04658 | 29.15252 |
| | | (ATPDR6/PDR6) | | | |
| GmaAffx.89473.2.A1_at | CK605927 | N/A | 23.84398 | 57.47345 | 72.94942 |
| Gma.1756.1.S1_x_at | BF324935 | Vacuolar processing enzyme gamma | 22.99967 | 15.05816 | 9.772365 |
| | | (GAMMA-VPE) | | | |
| Gma.1263.1.S1_at | AY144180 | PHOSPHOENOLPYRUVATE | 21.98572 | 10.48921 | 7.622626 |
| | | KINASE 2 (PPCK2) | | | |
| Gma.4359.1.S1_a_at | AI794712 | Thioredoxin domain 2 | 21.94326 | 11.43655 | 14.28961 |
| Gma.6642.1.A1_at | BE610052 | Armadillo/beta-catenin repeat family | 21.50123 | 10.42401 | 8.855311 |
| | | protein | | | |

A comparative analysis of the combined resistance reaction comparing 3, 6 and 9 dpi[Peking/PI 548402 ?PI
88788] to the pericycle[Peking/PI 548402 ? PI 88788] (baseline). FC Fold change, PV P value; com,
combined. A C|±1.5| fold cutoff and P B 0.05 was used. FDR was set to 12%. The probe set satisfied the
criteria set by the differential expression and FDR analyses. Full gene lists are Supplemental Table 10

Combined analysis-suppressed genes

| | | | | | |
|---|---|---|---|---|---|
| GmaAffx.91442.1.S1_at | CF805736 | Stress-induced protein SAM22 | −897.9834 | −692.1131 | −591.788 |
| Gma.6999.3.S1_s_at | CF921432 | Stress-induced protein SAM22 | −627.6774 | −425.2879 | −452.2152 |
| Gma.6999.2.S1_s_at | X60043 | Stress-induced protein SAM22 | −622.328 | −208.9094 | −155.8947 |
| Gma.6999.1.S1_s_at | AF529303 | Stress-induced protein SAM22 | −590.5102 | −202.3408 | −145.2682 |
| GmaAffx.91091.1.A1_at | CF806816 | OSMOTIN 34 (ATOSM34) | −573.473 | −691.8259 | −676.8448 |
| GmaAffx.90703.1.A1_at | CF809087 | Class III peroxidase | −542.0378 | −363.9944 | −272.1859 |
| Gma.6999.1.S1_x_at | AF529303 | Stress-induced protein SAM22 | −230.486 | −131.8197 | −112.7692 |
| GmaAffx.43551.1.S1_at | AW101224 | Epoxide hydrolase | −220.7934 | −78.91801 | −94.56628 |
| GmaAffx.7738.1.S1_s_at | BE058056 | N/A | −191.8086 | −126.3119 | −77.05466 |
| GmaAffx.90097.1.S1_at | CF805822 | ARABIDOPSIS THALIANA EXPANSIN | −182.8408 | −159.7812 | −102.9616 |
| | | A16 | | | |
| | | (ATEXPA16) | | | |
| GmaAffx.671.1.S1_at | CA785167 | ARABIDOPSIS THALIANA EXPANSIN | −173.251 | −136.7262 | −104.0401 |
| | | A16 | | | |
| | | (ATEXPA16) | | | |
| Gma.5785.1.S1_at | BI943300 | Endo-1,4-beta-glucanase | −163.7475 | −91.02507 | −71.93401 |
| Gma.8144.1.A1_at | BU548599 | Peroxidase | −156.2049 | −268.5381 | −223.8144 |
| Gma.413.1.S1_at | BQ611370 | Peroxidase 12 (PER12) | −147.7885 | −86.6731 | −54.48467 |
| Gma.4829.1.S1_at | AW309606 | Peroxidase | −136.0648 | −82.92677 | −38.63701 |
| GmaAffx.10710.1.S1_s_at | AW278629 | Pathogenesis-related protein PR-1 | −108.9026 | −61.13944 | −59.67152 |
| Gma.15760.1.S1_at | BE823195 | Histone H3 | −106.2577 | −54.28122 | −35.8704 |
| Gma.17993.1.S1_s_at | BQ628525 | ATOSM34 (OSMOTIN 34) | −102.6187 | −143.6977 | −141.4526 |
| GmaAffx.90861.1.S1_at | CF805971 | N/A | −100.1445 | −97.9008 | −110.9905 |
| GmaAffx.65048.1.S1_s_at | BQ628412 | N/A | −98.83867 | −78.19184 | −65.50682 |
| Gma.3604.1.S1_at | AW349604 | Caffeoyl-CoA 3-0-methyltransferase | −97.15329 | −131.8877 | −91.56036 |
| Gma.6327.1.S1_s_at | BM731752 | Senescence-associated protein | −91.85182 | −70.45923 | −71.3001 |
| Gma.16709.1.S1_s_at | BQ742929 | Cytochrome P450 | −90.20964 | −70.88486 | −67.98326 |
| Gma.1326.1.S1_at | CD397515 | Pectate lyase family protein | −87.18268 | −57.86263 | −51.77156 |
| GmaAffx.31985.1.S1_at | BI320384 | Arabidopsis thaliana chitinase class | −82.44353 | −68.22105 | −84.34828 |
| | | IV (ATEP3) | | | |
| Gma.13110.1.S1_at | CD394837 | ARABIDOPSIS THALIANA | −78.24623 | −46.97824 | −36.09789 |
| | | EXPANSIN A1 | | | |
| | | (ATEXPA1) | | | |
| GmaAffx.90390.1.S1_at | CF806115 | Peroxidase 12 (PER12) | −76.10898 | −63.17044 | −45.64938 |
| GmaAffx.42893.1.A1_at | BU549612 | Short-chain dehydrogenase/reductase | 76.05193 | −54.56057 | −42.04273 |
| | | (SDR) | | | |
| GmaAffx.93392.1.S1_s_at | CF807760 | Disease resistance-responsive family | 75.12781 | −56.15482 | −47.10637 |
| | | protein | | | |
| Gma.9228.1.S1_at | CD417435 | Arabinogalactan-protein | −72.61606 | −44.5325 | −34.99915 |
| Gma.2523.1.S1_s_at | CA852440 | R 14 protein | −71.60612 | −91.03295 | −61.58156 |
| Gma.15907.1.A1_s_at | CD407154 | Leucine-rich repeat protein | −69.31699 | −53.50557 | −52.53084 |
| Gma.15715.1.S1_at | CD403744 | N0D26-like intrinsic protein 1;2 | −68.95551 | −51.05334 | −52.08829 |
| | | (NIP1;2/NLM2) | | | |
| Gma.576.1.S1_at | BM732317 | Invertase/pectin methylesterase | −67.62666 | −52.36145 | −43.47715 |
| | | inhibitor | | | |
| GmaAffx.93214.1.S1_at | CF808939 | Cinnamate 4-hydroxylase | −66.4193 | −77.81764 | −57.90429 |
| Gma.8401.1.A1_at | BU547972 | Cytochrome P450, family 71, | −65.65226 | −72.91756 | −86.79321 |
| | | subfamily B, polypeptide | | | |
| | | 34 (CYP71B3) | | | |
| GmaAffx.89772.10.A1_s_at | CK606097 | Stress-induced protein 5AM22 | −64.95094 | −46.49229 | −80.43811 |
| Gma.2833.1.S1_s_at | AF202731 | BASIC CHITINASE (ATHCHIB) | −64.46766 | −47.3571 | −38.70762 |
| Gma.7436.1.S1_at | BU547698 | PLANTACYANIN | −63.56152 | −74.54568 | −57.79718 |
| GmaAffx.666.1.S1_at | BM143164 | Extracellular dermal glycoprotein | −61.81902 | −47.06001 | −10.94365 |
| | | (EDGP) | | | |
| GmaAffx.91087.1.S1_s_at | CF806812 | Endo-1,4-beta-glucanase | −60.08572 | −34.73255 | −33.00312 |
| Gma.9164.1.S1_at | CA802402 | Histone H4 | −56.8739 | −32.03909 | −26.51658 |
| Gma.4185.1.S1_at | BQ742395 | ARABIDOPSIS THALIANA EXPANSIN- | −56.60563 | −48.58276 | −40.16978 |
| | | LIKE B1 | | | |
| | | (ATEXLB1) | | | |
| Gma.413.1.S1_s_at | BQ611370 | Peroxidase 12 (PER12) | −55.98323 | −41.66081 | −29.62947 |
| Gma.7631.1.A1_at | AW309495 | Malate dehydrogenase/ | −55.34567 | −68.28899 | −46.8821 |
| | | NADP-MALIC ENZYME 3 | | | |
| | | (ATNADP-ME3) | | | |
| Gma.154.1.S1_at | Y10490 | Cytochrome P450, family 71, | −52.86155 | −63.53077 | −86.40897 |
| | | subfamily B, polypeptide 34 | | | |
| | | (“CYP71B34) | | | |
| Gma.876.1.S1_at | L78163 | Peroxidase | −52.25232 | −54.85669 | −28.92915 |
| GmaAffx.54524.1.S1_at | BU551371 | Caffeoyl-CoA 3-0-methyltransferase | −50.93016 | −28.59014 | −21.32015 |
| | | (CCoAMT) | | | |
| Gma.4434.1.S1_at | BE659015 | ARABIDOPSIS BLUE-COPPER- | −50.29084 | −47.34821 | −43.85539 |
| | | BINDING | | | |
| | | PROTEIN (ATBCB) | | | |
| Gma.3713.1.S1_s_at | BI469834 | Trypsin_and_protease_inhibitor_family_protein/ | −43.4449 | −61.40882 | −43.33708 |
| | | Kunitz_family_protein | | | |
| Gma.5590.1.S1_a_at | BE822176 | 3-Deoxy-c-arabino- | −33.39936 | −54.02632 | −44.45494 |
| | | heptulosonate_7- | | | |
| | | phosphate_synthase | | | |
| GmaAffx.75992.1.S1_at | BU548101 | N/A | −31.54975 | −50.64437 | −38.10328 |

A comparative analysis of the combined resistance reaction comparing 3, 6 and 9
dpi[Pekin_{g}/PI 548402 + PI 887881 to the peliCyCle_{[}p_{ekingim} 548402 + PI 887881 (baseline). FC
Fold change, PVP value, corn combined. A >1±1.5I fold cutoff and P < 0.05 was used. FDR
was set to 12%. The probe set satisfied the criteria set by the differential expression and
FDR analyses. Full gene lists are Supplemental Table 10

Histology

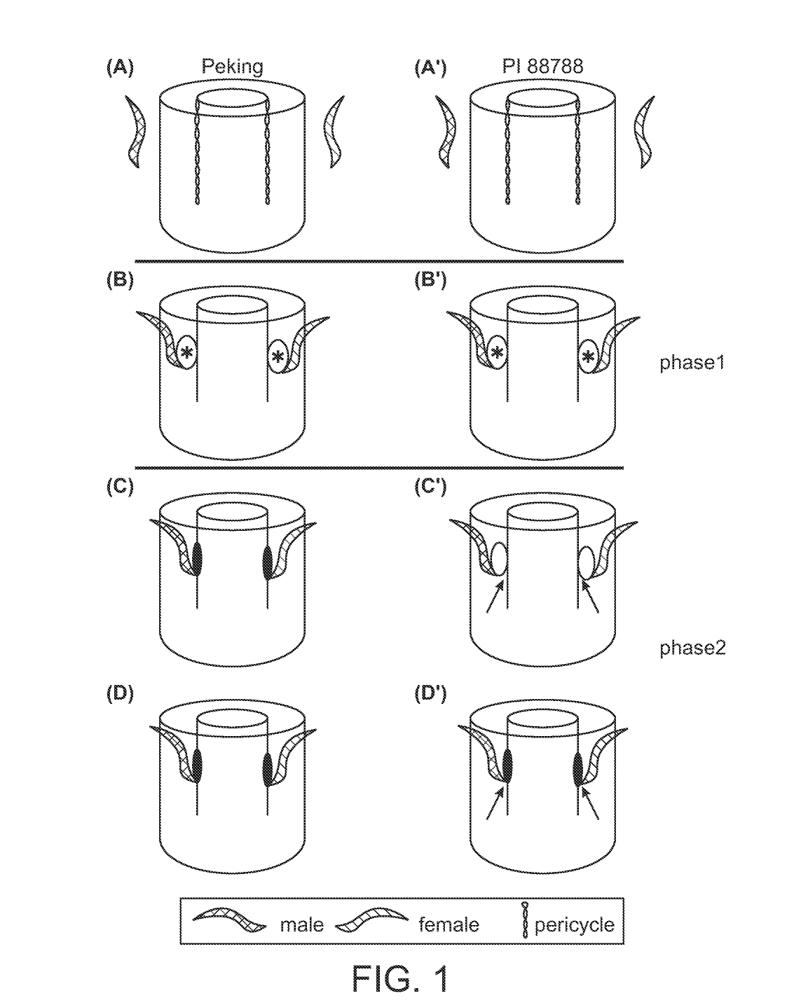

The resistant reaction at the syncytium undergoes two phases during its development that leads to mortality of the nematode (FIG. 1).

As will be appreciated from the figures, FIG. 1 shows G. max_{[Peking/PI 548402}] and G. max_{[PI 88788]} resistant reactions. FIG. 1a, and 1a′ depict male (left) and female (right) pre-infective J2 (pi-J2) nematodes migrate toward the root. FIGS. 1b and 1b′ show the infective J2 (i-J2) nematodes burrow into the root and migrate toward the root stele, typically selecting a pericycle or neighboring cell as the feeding site initial (FSi) and create a syncytium (white asterisk). The earlier stages of syncytium development (between 1 an 4 dpi) are similar between G. max_{[Peking]} and G. max_{[PI 88788]}-type of resistant reactions. FIG. 1c shows that in the G. max_{[Peking]}-type, a rapid and potent resistant reaction occurs by the formation of a necrotic region that surrounds the syncytium (black oval, white arrow) by 4 dpi. As will be appreciated from FIG. 1c′, in the G. max_{[PI 88788]}-type of resistance reaction, a slower response, characterized initially by nuclear degeneration within the syncytium (dark blue oval, white arrow), occurs by 5 dpi. In FIG. 1d, the G. max_{[Peking]}-type of resistant reaction is characterized by the later stages of syncytium degeneration (black oval, white arrow) at 7 dpi. Finally, in FIG. 1d′, the G. max_{[PI 88788]}-type of resistant reaction is characterized by the later stages of syncytium degeneration as the cytoplasm degrades (black oval, white arrow) at 10 dpi. (Timing of stages adapted from Endo 1965; Riggs et al. 1973 Lauritis et al. 1983; Kim et al. 198 7.)

The first phase (phase 1) is a parasitism phase whereby the nematode infects a cell and establishes the initial stages of syncytium development. The second phase (phase 2) is the resistance phase whereby syncytia collapse and cease to function. The parasitism phase is prolonged during a susceptible reaction, presumably by overriding the resistance phase. That activity results in a compatible interaction with the G. max genotype. Histological examination of syncytia is aided by the safranin Fast Green staining procedure (Sass 1958; Ross 1958; Endo 1965; Klink et al. 2005, 2007a, b, 2009a, 2010a, b). Safranin is a regressive stain, known to preferentially stain lignified, suberinized and cutinized tissues as well as staining chromosomes and nucleoli red. In contrast, the progressive counterstain Fast Green is known to preferentially stain cytoplasm and cellulosic cell walls. Histological examination of roots used in the analyses demonstrates that G. max_{[Peking/PI 548402]} and G. max_{[PI 88788]} roots are infected with (H. glycines_{[NL1-RHg/HG-type 7])} at 3 dpi (FIG. 2a, b), 6 dpi (FIG. 2c, d) and 9 dpi (FIG. 2e, f), respectively. FIG. 2 shows FIG. 2 Histological responses of G. max_{[Peking/PI 548402]} and G. max_{[PI 88788]} roots to H. glycines infection during their resistant reactions. a G. max_{[Peking/PI 548402]} at 3 dpi. b G. max_{[PI 88788]} at 3 dpi. c G. max_{[Peking/PI 548402]} at 6 dpi. d G. max_{[PI 88788]} at 6 dpi. e G. max_{[Peking/PI 548402]} at 9 dpi. f G. max_{[PI 88788]} at 9 dpi. Arrow, nematode, red line, perimeter of syncytium.

The walls of cells undergoing the parasitism stage appear to stain for cellulose in both G. max_{[Peking/PI 548402]} (FIG. 2a) and G. max_{[PI 88788]} (FIG. 2b). In contrast, the walls of the resistance phase of the resistant reaction appear to stain preferentially for lignin, suberin and/or cutin in G. max_{[Peking/PI 548402]} (FIG. 2c, e) and G. max_{[PI 88788]} and (FIG. 2e, f). This staining characteristic is also observed by Ross (1958), Endo (1965) and Klink et al. (2007a, b, 2009a, 2010a, b). Identification of syncytia is a simple diagnostic that was applied to the LCM analyses (FIG. 3a, b). FIG. 3 shows a FIG. 3 A microdissected syncytium. a Before LCM; b after LCM. Red line, perimeter of the syncytium. Black arrow, head of nematode, white arrows, microdissected syncytium.

Direct Comparison of G. max_{[Peking/PI 548402]} to G. max_{[PI 88788]} Syncytium Gene Expression

Prior analyses have identified genes that are induced or suppressed in the syncytia of G. max_{[Peking/PI 548402]} (Klink et al. 2007a, 2009a) and G. max_{[PI 88788]} (Klink et al. 2010a) undergoing their respective resistant reactions. However, it is known that the (H. glycines_{[NL1-RHg/HG-type 7])} population elicits two distinctly different resistant reactions in the G. max_{[Peking/PI 548402]} and G. max_{[PI 88788]} genotypes as it attempts to develop a functional syncytium. Thus, from the prior analyses, a gap in knowledge is the direct cross comparison of the G. max_{[Peking/PI 548402]} and G. max_{[PI 88788]} resistant reactions. The aim of the direct comparative analyses is examining relative levels of gene activity present in syncytia of the potent and rapid resistant reaction of G. max_{[Peking/PI 548402]} as compared to the potent but prolonged resistant reaction of G. max_{[PI 88788]} genotype (base line). Since G. max_{[PI 88788]} is used as the base line, the output for all of the experiments presented in the analysis is relative expression for G. max_{[Peking/PI 548402]}.

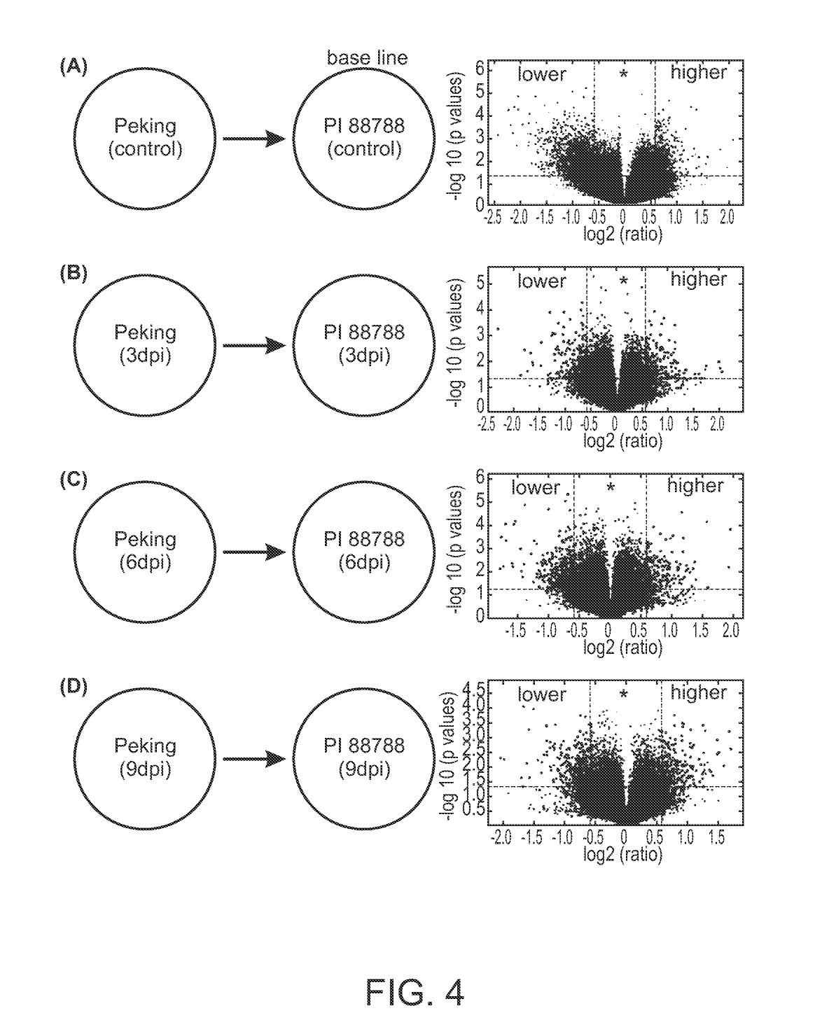

The first set of experiments compare relative levels of gene expression in pericycle and surrounding cells prior to infection (FIG. 4a) and from microdissected syncytia at the 3 (FIG. 4b), 6 (FIG. 4c) and 9 dpi (FIG. 4d).

FIG. 4 shows volcano plots depicting relative gene expression. To the left of the volcano plot is a graphic depicting the comparison being made. The gene expression of G. max_{[PI 88788]} is the base line of the comparisons. Therefore, expression is presented in terms of relative levels in G. max_{[Peking/PI 548402]}. a The G. max_{[Peking/PI 548402]} pericycle isolated from uninoculated roots vs. G. max_{[PI 88788]} pericycle (baseline) that was isolated from uninoculated roots. b 3 dpi G. max_{[Peking/PI 548402]} syncytium vs. 3 dpi G. max_{[PI 88788]} syncytium. c 6 dpi G. max_{[Peking/PI 548402]} syncytium vs. 6 dpi G. max_{[PI 88788]} syncytium. d 9 dpi G. max_{[Peking/PI 548402}] syncytium vs. 9 dpi G. max_{[PI 88788]} syncytium. A C|±1.5| fold cutoff and P B 0.05 with a FDR set at 12% was used for the analyses. Genes with higher relative levels of expression (dark blue, upper right quadrant) and genes with lower relative levels of expression (dark blue, upper left quadrant) in G. max_{[Peking/PI 548402]} are presented. Genes with no statistically significant differences in expression used later in the combined analyses are denoted by an asterisk (*)

As expected, the experiments reveal that much of the measurable relative levels of gene expression occurring between the G. Max_{[Peking/PI 548402]} and G. max_{[PI 88788}] genotypes is similar. Therefore, the experiments show the robustness of the relative expression data for thousands of genes in two different genotypes. However, a smaller amount of gene expression is found to be significantly different between the G. max_{[Peking/PI 548402]} and G. max_{[PI 88788]} genotypes. The observation reveals that amplitude differences in expression exist between the potent and rapid G. max_{[Peking/PI 548402]} resistant reaction and the potent but prolonged resistant reaction of G. max_{[PI 88788]}.

G. max_{[Peking/PI 548402]} pericycle cells have amplified levels of genes pertaining to defense pathways prior to infection

The direct comparative analyses of the pericycle and surrounding cells isolated from uninfected roots identified a probe set for the differentially expressed in response to arachidonic acid 1 gene (DEA1 [Gm-DEA1]) (CA850542) to measure the greatest difference in relative gene expression (224.19-fold) when comparing G. max_{[Peking/PI 548402]} to G. max_{[PI 88788}]. A second probe set measuring higher relative levels of gene expression was a protease inhibitor (BU082252) (68.28-fold). Other probe sets with high relative expression levels greater than 20-fold in G. max_{[Peking/PI 548402]} include 3 polygalacturonidases (CF808466, CD414773, AF128266), an R-gene (BI785070), 2 lipoxygenases (CD409280, BM092012), EMBRYO DEFECTIVE 1374 (CD401715), Zwille-like protein (BG651396) and ACC oxidase (BE440266) (Table 1; Supplemental Table 1). Probe sets measuring relatively lower levels of gene expression in G. max_{[Peking/PI 548402]} were also identified (Supplemental Table 1).

G. max_{[Peking/PI 548402]} syncytia have amplified levels of genes pertaining to defense pathways during the resistant reaction

Expression analyses followed that compare G. max_{[Peking/PI 548402]} syncytium gene expression directly to G. max_{[PI 88788]} syncytium gene expression at the 3 dpi (FIG. 4b; Supplemental Table 2), 6 dpi (FIG. 4c; Supplemental Table 3) and 9 dpi time points (FIG. 4d; Supplemental Table 4). The analyses identify differences in gene expression both prior to and during both phase 1 and phase 2 of the resistant reactions. Pathway analyses are presented in the next section.

Pathway analyses identify amplified levels of genes in G. max_{[Peking/PI 548402]} Syncytia as compared directly to G. max_{[PI 88788]} at 3 dpi as Supplemental Data Link: 3 dpi

Pathway analysis. Similar direct comparative analyses of the G. max_{[Peking/PI 548402]} and G. max_{[pJ 88788]} genotypes were expanded to the 6 and 9 dpi time points.

Pathway analyses identify amplified levels of genes in G. max_{[Peking/PI 548402]} Syncytia as compared directly to G. max_{[PI 88788]} at 6 dpi

Pathway analyses were done that directly compared 6 dpi G. max_{[Peking/PI 548402}] syncytia to 6 dpi G. max_{[PI 88788]} syncytia. At 6 dpi, amplified levels of genes in the antho-cyanin biosynthesis pathway (Supplemental FIG. 3A), glycosphingolipid (Supplemental FIG. 3B) and high marmose type N-glycan biosynthesis (Supplemental FIG. 3C) were observed. In contrast, suppressed levels of brassinosteroid biosynthesis (Supplemental FIG. 3D), fatty acid biosynthesis (Supplemental FIG. 3E) and phenylpropanoid pathways (Supplemental FIG. 3F) were observed. All 6 dpi direct comparison pathways can be found at the Supplemental Data Link 6 dpi pathway analysis.

Pathway analyses identify amplified levels of genes in G. max_{[Peking/PI 548402]} Syncytia as compared directly to G. max_{[PI 88788]} at 9 dpi

Pathway analyses were done that directly compared 9 dpi G. max_{[Peking/PI 548402}] syncytia to 9 dpi G. max_{[PI 88788]} syncytia. At 9 dpi, amplified levels of genes in the anthocyanin biosynthetic pathway (Supplemental FIG. 4A), alpha linoleic acid metabolism pathway (Supplemental FIG. 4B), fatty acid elongation in mitochondria pathway (Supplemental FIG. 4C), glycosphingolipid biosynthesis pathway (Supplemental FIG. 4D), Fatty acid metabolism pathway (Supplemental FIG. 4E), high mannose type N-glycan biosynthesis pathway (Supplemental FIG. 4F), peptidoglycan biosynthesis pathway (Supplemental FIG. 4G), metabolism of xenobiotics by cytochrome P450 (Supplemental FIG. 4H), ubiquinone and terpenoid-quinone biosynthesis pathways (Supplemental FIG. 4I). Relatively lower levels of the brassinosteroid biosynthesis pathway (Supplemental FIG. 4J) and carotenoid biosynthetic pathways (Supplemental FIG. 4K) were observed. While components of important defense pathways are induced in either G. max_{[Peking/PI 548402]} or G. max_{[PI 88788]} as compared to their respective pericycle and surrounding cells, the observations demonstrate further that amplitude differences exist between the G. max_{[Peking/PI 548402}] genotype as compared to G. max_{[PI 88788]}. All 9 dpi direct comparison pathways can be found at the Supplemental Data Link: 9 dpi pathway analysis.

Differences in relative levels of gene expression are sustained throughout the resistant reaction of G. max_{[Peking/PI 548402]} or G. max_{[PI 88788]} during the 3, 6 and 9 dpi time points.

The aforementioned analyses were time point specific and did not address whether the amplitude differences in gene expression spanned the duration of the resistant reactions of G. max_{[Peking/PI 548402]} or G. max_{[PI 88788]}. Analyses were done to compare the relative gene expression levels occurring between G. max_{[Peking/PI 548402]} and G. max_{[PI 88788]} at the 3, 6 and 9 dpi time points. This period represents the time course of infection. In these highly stringent analyses, to have sustained modulated gene expression, the criteria for amplified (consistently higher) or attenuated (consistently lower) levels of expression are satisfied when the probe set measures expression in a similar manner in the 9 biological replicates across the two genotypes (i.e., 3 biological replicates×3 time points×2 genotypes; FC>1.5, P<0.05 and FDR<10%) for a total of 18 arrays. As expected, most probe sets did not measure differences in the level of expression between the two genotypes at all time points due to the stringency of the analysis. However, the analyses resulted in the identification of 93 probe sets measuring consistently and statistically significant differences in relative levels of gene expression in G. max_{[Peking/PI 548402]} as compared to the G. max_{[PI 88788]} genotype at all time points. Of those 93 probe sets 57 have higher relative levels of gene expression in G. max_{[Peking/PI 548402]} at the 3, 6 and 9 dpi time points (Table 2; Supplemental Table 5). The analysis also identified 27 probe sets that measure lower relative levels of gene expression at the 3, 6 and 9 dpi time points in G. max_{[Peking/PI 548402]} as compared to G. max_{[PI 88788]} (Table 2; Supplemental Table 5). The analyses demonstate that while much of the relative levels of gene expression are similar between the G. max_{[Peking/PI 548402]} and G. max_{[PI 88788]} genotypes during infection, a smaller number of probe sets measure relative gene expression levels that are consistently and statistically higher or lower in G. max_{[Peking/PI 548402]} as compared directly to the G. max_{[PI 88788]} genotype, but only after infection of the root cells by H. glycines.

The identification of constitutive differences in relative levels of gene expression that are present between the G. max_{[Peking/PI 548402]} and G. max_{[PI 88788]} genotypes

The prior analyses identified genes that initially had similar levels of gene expression in the control cell populations of both G. max_{[Peking/PI 548402]} and G. max_{[PI 88788]} genotypes. These similar levels were followed by modulations in gene activity that could be measured between G. max_{[Peking/PI 548402]} and G. max_{[PI 88788]} genotypes only at 3, 6, 9 dpi. The analyses presented here included the pericycle and surrounding cell (control) time point samples with the 3, 6 and 9 dpi time points. The analyses identify a different pool of probe sets that measure differences in relative gene expression on 24 arrays (i.e., 3 biological replicates×time points×2 genotypes; FC>1.5, <0.05 and FDR<10%), representing all the time points. The analysis was done to identify the list of genes that had different levels of expression that were attributed to the G. max_{[Peking/PI 548402]} and G. max_{[PI 88788]} geno-types. Since gene expression was always different both prior to and after infection, the relative level of expression likely was intrinsic to the genotype and not due to infection by H. glycines. The analyses identified 25 probe sets that measure consistently and statistically significant higher relative levels of gene expression in G. max_{[Peking/PI 548402]} as compared to G. max_{[PI 88788]} across the 4 time points (Table 3; Supplemental Table 6). The analysis also identified 5 additional probe sets that measured statistically significant lower relative levels of gene expression in G. max_{[Peking/PI 548402]} as compared to G. max_{[PI 88788]} at the 4 time points (Table 3; Supplemental Table 6).

Commonalities of the G. max_{[Peking/PI 548402]} and G. max_{[PI 88788]} resistant reactions

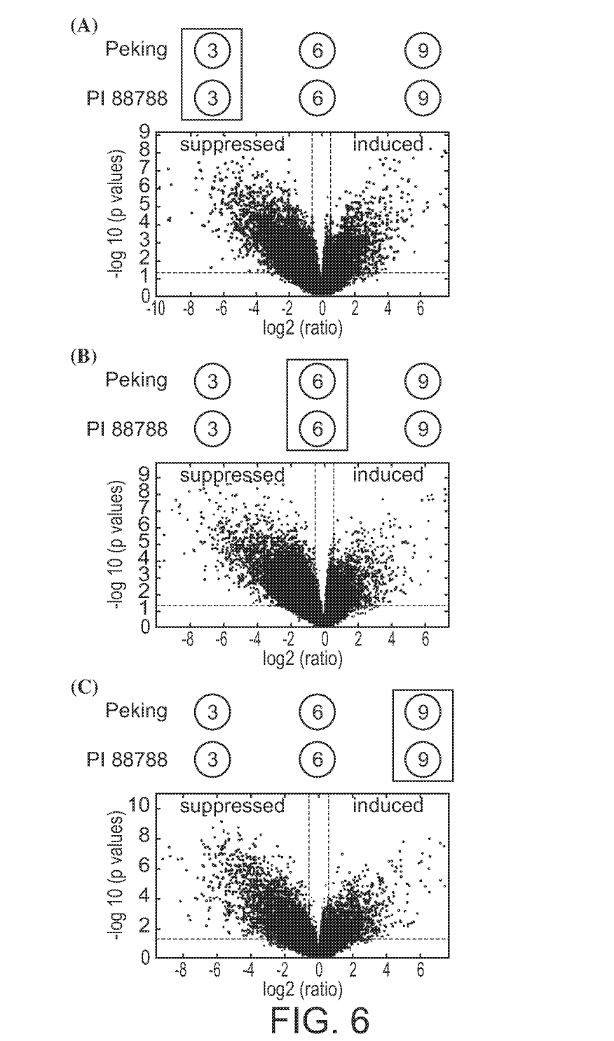

The previous analyses identified probe sets measuring different relative levels of gene expression occurring between G. max_{[Peking/PI 548402]} and G. max_{[PI 88788]}. However, the volcano plots demonstrate that a substantial number of probe sets are measuring similar relative levels of gene expression when directly comparing G. max_{[Peking/PI 548402]} and G. max_{[PI 88788]} (FIG. 4). The probe sets that measure similar levels of relative gene expression between G. max_{[Peking/PI 548402]} and G. max_{[PI 88788]} became the focus of further investigations. In these experiments, combined dated from G. max_{[Peking/PI 548402]} and G. max_{[PI 88788]} are used to examine gene expression that is common to the two genotypes. The analyses are referred to as combined analyses because they combine the gene expression data of G. max_{[Peking/PI 548402]} and G. max_{[PI 88788]} at each time point. The analyses result in the identification of probe sets that measure induced or suppressed levels of gene expression at each time point as compared to pericycle and the surrounding cells. The analyses are unlike the previous experiments that were designed to measure relative expression levels between the two genotypes (FIG. 4). In the combined analyses, all probe sets that measured statistically significant differences in relative levels of gene expression between G. max_{[Peking/PI 548402]} and G. max_{[PI 88788]} were eliminated from further analyses. The combined analyses use the 3 replicates from G. max_{[Peking/PI 548402}] and 3 from G. max_{[PI 88788]} from each time point, totaling 6 replicates. The combined replicates are used to compare the 3, 6 or 9 dPi[Peking/pI 548402±PI 88788] samples to the pericycle[Peking/PI 548402±PI 88788] control (FIG. 6). FIG. 6 Volcano plots comparing differential gene expression of the 3, 6 or 9 dpi[Peking/PI 548402+PI 88788] combined syncytium samples to the pericycle[Peking/PI 548402+PI 88788] combined sample. The pink box depicts the sample types under study. A C|±1.5| fold cutoff and P B 0.05 with a FDR set at 12% was used for the analyses. Induced genes, dark blue, upper right quadrant. Suppressed genes, dark blue, upper left quadrant. a 3 dpi[Peking/PI 548402+PI 88788] combined resistant syncytium sample as compared to the pericycle[Peking+PI 88788] combined sample. b 6 dpi[Peking/PI 548402+PI 88788] combined syncytium sample as compared to the pericycle[Peking/PI 548402+PI 88788] combined sample. c 9 dpi[Peking/PI 548402+PI 88788] combined syncytium sample as compared to the pericycle[Peking/PI 548402+PI 88788] combined sample

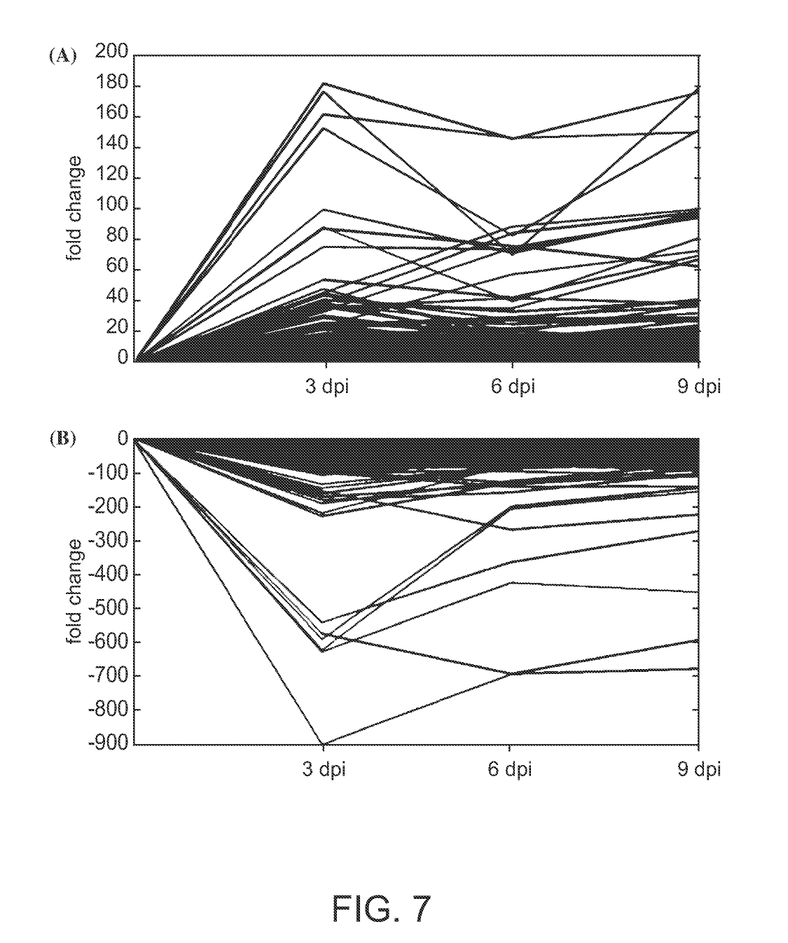

In the first combined analysis, the 3 dPi[Peking/PI 548402±PI 88788] Syncytium is compared to pericycle [Peking/PI 548402±PI 88788] (FIG. 6a). The analysis identified 1983 probe sets that measure induced levels of gene expression and 4404 probe sets measuring suppressed levels of gene expression in the 3 dPi[Peking/pI 548402±PI 88788] syncytium sample (Supplemental Table 7). In the second combined analysis, the 6 dPi[Peking/pI 548402±PI 88788] sample is compared to the pericycle[Peking/PI 548402±PI 88788] sample (FIG. 6b). The analysis identified 2118 probe sets that measure induced levels of gene expression and 5387 probe sets that measure suppressed levels of gene expression in the 6 dPi[Peking/pI 548402±PI 88788] syncytium sample (Supplemental Table 8). In the third combined analysis, the 9 dPi[Peking±PI 88788] sample is compared to the pericycle[Pekin_{g}/PI 548402±PI 88788] sample (FIG. 6c). The analysis identified 2739 probe sets that measure induced levels of gene expression and 1639 probe sets that measure suppressed levels of gene expression in the 9 dpi[Peking/PI 548402±PI 88788] syncytium sample (Supplemental Table 9). The prior combined analyses, examining individual time points, are examined further to identify probe sets consistently measuring induced or suppressed levels of gene expression throughout the infection process. Time course analyses of the combined samples identified probe sets that measure induced or suppressed levels of gene expression across all time points (3, 6 and 9 dpi[_{Peking/PI 548402±PI 88788]} as compared to the pericycle_{[Peking/PI 548402±PI 88788]} (FIG. 7; Supplemental Table 10).

FIG. 7 is a line graph depicting genes that are induced or suppressed in syncytium samples at the 3, 6 and 9 dpi time points as compared to pericycle samples in both G. max_{[Peking/PI 548402]} and G. max_{[PI 88788]}. A C|±1.5| fold cutoff and P B 0.05 with a FDR set at 12% was used for the analyses. a Induced genes. b Suppressed genes

The analysis identified 305 probe sets that measure induced levels of gene expression during the 3, 6 and 9 dpi time points. Probe sets measuring induced gene expression at or greater than an arbitrarily selected cutoff of 20-fold in at least one of the 3 time points and having statistically significant levels of induced gene expression at the other two time points are presented (Table 4). In contrast, there are 720 probe sets that measure suppressed levels of gene expression at all three time points. Probe sets measuring suppressed levels of gene expression of less than an arbitrarily selected cutoff of −50-fold in at least one of three time points are presented (Table 5; Supplemental Table 10).

Discussion

Resistance in plants to pathogens is a complex and multifaceted process, involving hormones such as jasmonic acid and salicylic acid, resistance proteins (R-genes), small RNAs, enzymatic processes as well as secondary metabolites such as terpenoids and stilbenoids (among many others). The differences in metabolic activity of the cells under pathogen attack are accompanied by subtle but important differences in the cellular architecture at the interface between plants and their parasites. For example, in Triticum aestivum, changes in the wheat leaf cuticle are associated with resistance to the Hessian fly, Mayetiola destructor (Say) (Kosma et al. 2010). In G. maxp_{ekingi}, cytological changes have also been associated with H. glycines infection that include the formation of CWAs (Kim et al. 1987; Kim and Riggs 1992). An understanding of the resistant reaction has been aided by the use of cytological stains such as safranin, a stain that preferentially stains lignin, suberin and cutin. These secondary metabolites have all been shown to be associated with resistance by plants to pathogens. However all of the genes involved in the processes remain to be identified.

In G. max, mapping efforts have been published since 1960 (Caldwell et al. 1960; reviewed in Concibido et al. 2004). More recently, sequencing efforts of regions spanning the resistant loci have been performed, resulting in the identification of and subsequent supposition that the genes responsible for H. glycines resistance were R-genes of the leucine rich repeat (LRR) class. However, in at least one case, the R-gene proposed to be responsible for the resistance of G. max to H. glycines a decade ago at the rhg1 locus does not function in the process (Melito et al. 2010). It is unlikely that the few resistant plant introductions contain all of the genes or have lesions in all of the genes involved in the resistance process. Thus, alternative approaches like the transcriptomic analyses of the syncytium undergoing the different forms of the resistant reaction presented here may help in identifying the genes through their expression patterns. This is an important tool especially if no lesions exist in the genes regulating the process.

The interaction between soybean and the soybean cyst nematode is a very specific reaction with the outcome regulated by both the genotype of the plant and the population of the nematode. Prior analyses have shown that a single G. max genotype, max[Peking/PI 548402], responds differently to two distinct populations of H. glycines during infection (Klink et al. 2007a, b, 2009a, 2010b). The studies reveal that whole H. glycines-infected roots of G. max_{[Peking, PI 548402]} are undergoing modulations in gene expression activity by 12 h post infection (hpi) as the roots develop a resistant (H. glycines_{[NL1-RHg/HG-type 7]}-infected) or a susceptible (H. glycines[TN8/HG-type 1.3.6.7]-infected)-infected) reaction (Klink et al. 2007b). It is known that G. max_{[Peking/PI 548402]} makes syncytia that are indistinguishable from each other in H. glycines_{[NL1-RHg/HG type 7]} and H. glycines_{[TN8/HG type 1.3.6.7]} infected roots at 3 dpi (Klink et al. 2007a, b, 2009a, 2010b), consistent with numerous cytological and ultra-structural studies (Endo 1965; Riggs et al. 1973; Acido et al. 1984; Kim et al. 1987). It is unclear what factor(s) underlie the inability of H. glycines_{[NL1-RHg/HG type 7]} to sustain a susceptible reaction in G. max_{[Peking/PI 548402]} or G. max_{[PI 88788]}.

Complicating the issue is the ability of H. glycines_{[NL1-RH/g HG-type 7]} to successfully infect other soybean genotypes such as the susceptible G. max_{[Kent/PI 548586]} (Al-khan:¹⁹f et al. 2006), G. max_{[MiniMax/PI 643148]} (Klink et al. 2008) or G. max_{[Williams 82/PI 518671]} (Klink et al. 2009b). However, experiments comparing gene expression of syncytia in G. max_{[Peking/PI 548402]} during resistant (H. glycines_{[NL1-RHg/HG-type7]}-infected) or susceptible (H. glycines_{[TN8/HG-type 1.3.6.71}-infected) reactions at 3 dpi reveal modulations in gene activity between the two reaction types (Klink et al. 2007a, 2009a, 2010b).

Experiments have demonstrated that H. glycines can alter gene expression in a normally resistant G. max genotype to accommodate its infection and pathogenicity (Mahalingham et al. 1999; Klink et al. 2007a, b, 2009a, 2010b). The question remained as to how these different nematode populations are equipped to accomplish a susceptible reaction in an otherwise resistant genotype. Recent transcriptomic experiments examining H. glycines_{[NL1-RHg/HG-type 7]} and H. glycines_{[TN8/HG-type 1.3.6.7]} revealed that the two nematode populations were indeed different, even before they infected the roots of G. max_{[Peking/PI 548402]} (Klink et al. 2009b). Some of these H. glycines genes experiencing different levels of gene expression are putative parasitism genes (Klink et al. 2009b). This discovery demonstrated that amplitude differences in putative parasitism genes accompany a compatible reaction as compared to an incompatible reaction. It remains to be demonstrated whether these amplitude differences contribute to a compatible reaction in an otherwise resistant soybean genotype. Differences at the DNA level have been observed for avirulent and virulent glycines populations as revealed by the use of 454 microbead sequencing (Bekal et al. 2008). The work reinforces evidence provided by transcriptomic analyses (Klink et al. 2009b) that genetic differences are present between avirulent and virulent H. glycines populations.

Studies have examined modulations in gene activity in various resistant G. max genotypes during infection by H. glycines race 3 (Mahalingham et al. 1999). Mahalingham et al. (1999) examined protein expression during the resistant reaction in three different G. max genotypes exhibiting resistance to H. glycines. The G. max genotypes used in the analysis were G. max_{[Peking]}, G. max_{[PI 88788]} and G. max_{[PI 437654]}. Thus, the study relates well to the work presented here. The study investigated polygalacturonase (PG) and polygalacturonase inhibitor protein (PG1P) expression in roots infected with race 3 and race 14. The race 3 used in Mahalingham et al. (1999) study functions in a similar way in their max_{[Peking]} and G. max_{[PI 88788]} genotypes as the glycines HG-type 7 population used in the analyses presented here. In those studies, race 3 would elicit a resistant reaction in G. max_{[}p_{eking]} and G. max_{[PI 437654]} and G. max_{[PI 88788]}. Race 14 would elicit a resistant reaction in G. max_{[PI 88788]} and G. max[pi_{437654]} and a susceptible reaction in G. max_{[}p_{eking]}. The studies demonstrated modulations in PG and PG1P proteins with amplified levels of PG in G. max_{[Peking]} as compared to G. max_{[PI 88788]} and G. max[pi_{437654]} (Mahalingham et al. 1999). While the induced and amplified levels were found in the susceptible reaction, it demonstrated that both induced and amplified expression do occur in different G. max genotypes infected with the same H. glycines population (Mahalingham et al. 1999) which is exactly what we observed in our genomics-based analyses.

Genotype-Specific Modulations in Gene Expression at the Site of the Resistant Reaction

A gap in knowledge from those experiments is how a single H. glycines population (i.e., NL1-RHg/HG-type 7) can elicit resistant reactions that are completely different at the cellular level in two different G. max genotypes. The advantage of procedures such as the LCM methodology is that the cells of interest can be purified to the exclusion of those not involved in the process. Subsequent bioinformatics analyses have allowed for the determination of whether the vast differences in cytology observed for the different resistant reactions at the site of infection for the G. max_{[Peking/PI 548402]}-type and G. max_{[PI 88788]}-type reactions are accompanied by diverse transcriptomic patterns. The analyses have also determined whether those differences in gene expression are imprinted into the root cells prior to infection or only occur after nematode infection. Alternatively, it is possible that such experiments would reveal only conserved patterns of expression that are accompanied by specific modulations in gene expression characteristic of each genotype that are occurring during the respective resistant reactions of G. max_{[Peking/PI 548402]} and G. max_{[PI 88788]}. The analysis presented here fills that gap in knowledge by examining resistant reactions in action in G. max_{[Peking/PI 548402]} and G. max_{[PI 88788}] to giyCitleS_{[NLi}_{_{—}}_{mig/HG}_{_{—}}_{type 7]}, locally at the syncytium. Many of the identified genes are not discussed here because they have been discussed in the individual analyses of G. max_{[Peking/PI 548402]} (Klink et al. 2007a, 2009a) and G. max_{[PI 88788]} (Klink et al. 2010a). We note that all genes identified in these analyses, while experiencing differences in expression level, may not relate to the resistant reaction in any way.

Gene expression in pericycle cells of G. max_{[}p_{eking]} is different to G. max_{[PI 88788]} prior to the introduction of H. glycines_{[ismi}_{_{—}}_{RHg/HG—type 7]} to the roots.

The first set of analyses compare G. max_{[Peking/PI 548402]} to G. max_{[PI 88788}] pericycle and surrounding cells, revealing differences in gene expression are present. The result demonstrates it is possible that determinants involved in resistance could be imprinted within the pericycle and surrounding cells (i.e., the nurse cell initials) prior to infection. One gene that experiences the largest difference in relative gene expression in G. max_{[Peking/PI 548402]} pericycle is DEAL. The G. max DEA1 cDNA (CA850542) was originally isolated from G. max_{[Peking/PI 548402}] roots infected with H. glycines_{[NLi}_{_{—}}_{mig/HG—type 7]}) at 2 and 4 dpi (N. W. Alkharouf and B. F. Matthews, unpublished data). In tomato, DEA1 exhibits organ-specific expression. DEA1 is highly expressed in roots, stems, and leaves (Weyman et al. 2006a). The DEA1 gene is induced by arachidonic acid (AA). AA is a polyunsaturated fatty acid molecule that is produced by various pathogens (i.e., Phytopthora infestans) and is known to trigger programmed cell death (PCD). Cell death has been observed in the syncytia of G. max_{[Peking/PI 548402]} (Kim and Riggs 1992). In other plant-pathogen interactions, AA is shown to be released from germinating P. infestans spores (Ricker and Bostock 1992) and can mimic the PCD response (Bostock et al. 1981; Bostock et al. 1986). The DEA1 primary amino acid sequence has a conserved, shared domain found in the eight-cysteine motif superfamily of protease inhibitors. The domain is also found in proteins such as alpha-amylase inhibitors, lipid transfer proteins and seed storage proteins (Weyman et al. 2006a). Reporter experiments involving normal protoplasts and protoplasts undergoing plasmolysis show that DEA1 is associated with the cell membrane (Weyman et al. 2006a). More recent experiments show that expression of DEA1 in a heterologous yeast system protects the yeast from freezing death (Weyman et al. 2006b). It may be that G. max homolog of DEA1 performs a role in the defense response at the site of infection. It may explain why G. max_{[Peking/PI 548402]} experiences rapid degradation of the syncytium during its resistance reaction while G. max_{[PI 88788]} experiences a prolonged localized response at the syncytium. The identification of Gm-DEA1 is consistent with anatomical studies revealing that nuclei degrade in all forms of the resistant reaction. The process initiates by the formation of masses of chromatin that later scatter and deteriorates within the degenerating cytoplasm (Kim and Riggs 1992). The observation of higher levels of Gm-DEAlin G. max_{[Peking/PI 548402]} earlier than what is found in G. max_{[PI 88788]} is consistent with its more rapid appearance of the resistance reaction. AA, functioning upstream of jasmonate signaling (Blee 2002) may provide a way to amplify the signal leading to the rapid and potent resistant reaction of G. max_{[Peking/PI 548402]} and pathway, linking knowledge of the involvement of jasmonate signaling in the resistance of plants to parasitic nematodes (Gao et al. 2008).

Numerous other genes involved in defense and signaling were also identified in pericycle and surrounding cell samples isolated from roots that were not yet exposed to glycines[NL1-RH_{g}/HG4_{y}pe 7]. The identification of EMB1374 (CD401715) implicates MAP-kinase signaling cascades that could be more active in G. max_{[Peking/PI 548402]} than in G. max_{[PI 88788]} even before infection occurs. A probe set with homology to the A. thaliana gene At4g26500 (NP 194380) measures higher relative gene expression levels in G. max_{[Peking/PI 548402]} pericycle control samples as well as syncytia microdissected from 3, 6 and 9 dpi roots. The CD401715 expressed sequence tag (EST) has homology to a gene known as embryo defective 1374 (EMB 1374). EMB 1374 is also known as ARAB1DOPSIS THALIANA SULFUR E, ATSUFE, CHLOROPLAST SULFUR E, CPSUFE and SULFUR E 1, SUFE1. The EMB1374 mutant was originally isolated in a genetic screen in A. thaliana for mutants that were embryo defective (Tzafrir et al. 2001; McElver et al. 2001). In A. thaliana, EMB1374 both interacts with and activates the cysteine desulfurases, AtSufS in plastids and AtNifS1 in mitochondria. Each of these activations is vital during embryogenesis. Dual localization of the EMB 1374 protein occurs in mitochondria and chloroplasts. EMB 1374 is involved in Fe—S cluster biogenesis in both mitochondria and plastids. Little was known about how the gene works in plants until recent experiments in A. thaliana demonstrated that EMB 1374 interacts with mitogen-activated protein kinase-1 (MPK1) and MPK16 (Popescu et al. 2008). Thus, EMB1374 may play a central role in a variety of important signaling cascades within plant cells. For example, MPK1 alone has been shown to be phosphorylated by the MPKactivating kinases (MKKs) 1, 3, 7 and 10 (Popescu et al. 2008). MPK1 phoshorylates the downstream transcription factor targets WRKY40 and WRKY62 (Popescu et al. 2008). WRKY genes are known to play important roles in developmental processes, defense responses against pathogens and senescence (Eulgem and Somssich 2007). Thus, the analysis identified genes that are present in higher levels in pericycle cells. However the differences in amplitude of some of those genes extended throughout the resistant reaction in G. max_{[Peking/PI 548402]}. The observation demonstrates that the soybean genotypes are fundamentally different in the cells that are involved in the resistant reaction. Some differences are observed prior to the association of nematodes with the root while some of those differences are limited to the period of infection and some exhibit differences in expression that are constitutive. Pathway analyses reveal amplified levels of genes involved in defense in the rapid and potent resistant of G. max_{[Peking/PI 548402]} as compared to G. max_{[PI 88788]}

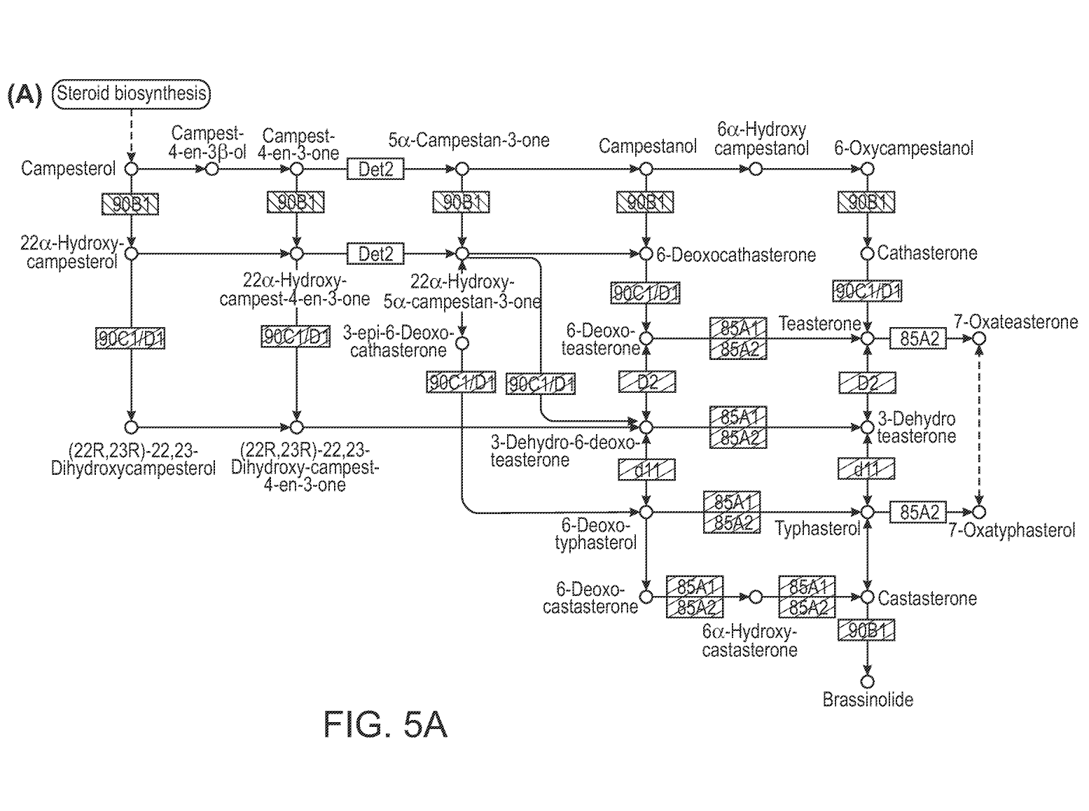

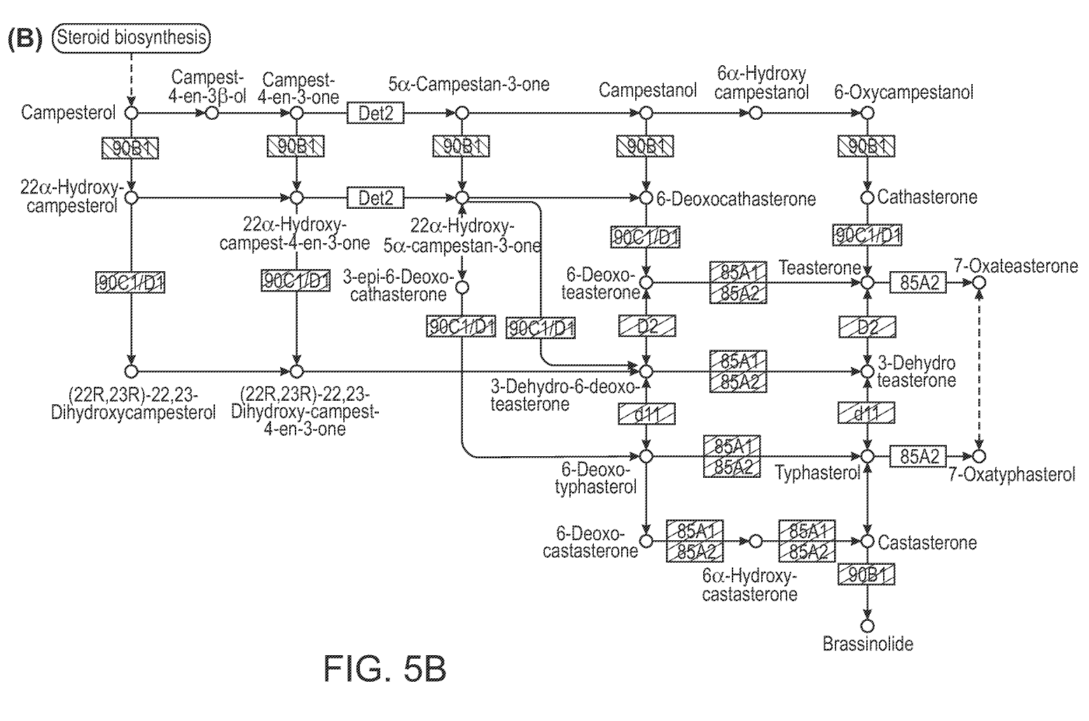

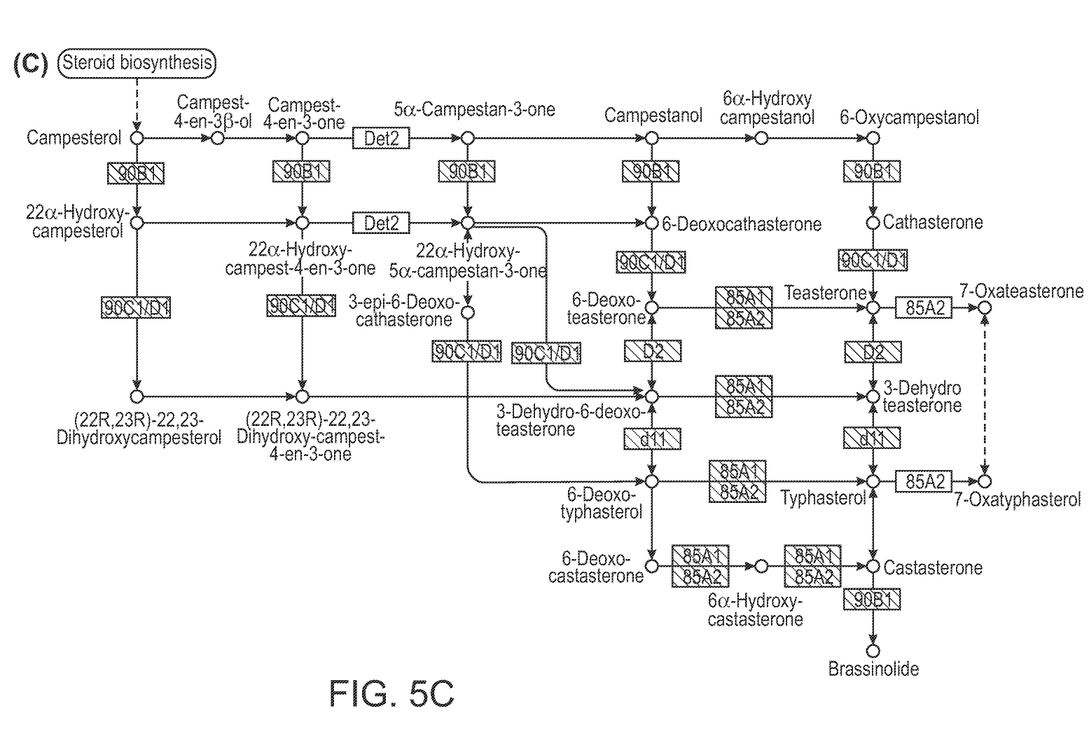

Customized pathway analyses tools have been developed and used to obtain a better understanding of genes having homology to those with known function. The work pro-vides a broader understanding of gene expression during the respective resistant reactions. However, it is noted that pathway analyses are done to the exclusion of many highly induced/suppressed amplified/attenuated genes that may be important to the resistant reaction. The initial analyses first compared both G. max_{[Peking/PI 548402]} and G. max_{[PI 88788]} to their respective pericycle cells. The analyses determined induced levels of genes pertaining to important aspects of defense or applicable to defense such as brassinosteroid signaling pathway (Nakashita et al. 2003; He et al. 2007; Chinchilla et al. 2007; Heese et al. 2007; Lu et al. 2010), some components of the fatty acid biosynthesis pathway (Kachroo et al. 2003, 2008; Chandra-Shekara et al. 2007; Reina-Pinto et al. 2009), glycolysis (Scheideler et al. 2002; Colebatch et al. 2002), components of the phenylpropanoid biosynthesis pathway (Cole 1984; Leszczynski et al. 1989; Edens et al. 1995; Wang et al. 2006) and ubiquinone and terpenoid-quinone biosynthesis pathways in the G. max_{[Peking/PI 548402]} and G. max_{[PI 88788]} genotypes. Some of the genes presented here were identified in prior analyses of G. max_{[Peking/PI 548402]} (Klink et al. 2009a) and G. max_{[PI 88788]} (Klink et al. 2010a). In contrast to the previous experiments, the analyses presented here determined whether certain pathways were both induced and amplified or suppressed and attenuated in their expression in one genotype in comparison to another. The analyses identified the induced and amplified nature of the brassinosteroid pathway in G. max_{[Peking/PI 548402]} as compared to G at 3 dpi (FIG. 5a-c). FIG. 5 shows a FIG. 5 Pathway analysis and comparison of the brassinosteroid biosynthesis pathway. a The brassinosteroid biosynthesis pathway in G. max_{[Peking/PI 548402]} 3 dpi syncytia gene expression as compared to G. max_{[Peking/PI 548402]} pericycle and surrounding cells. b. The brassinosteroid biosynthesis pathway in G. max[PI 88788] 3 dpi syncytia gene expression as compared to G. max[PI 88788] pericycle and surrounding cells. c The brassinosteroid biosynthesis pathway in G. max_{[Peking/PI 548402]} 3 dpi syncytia gene expression as compared to the brassinosteroid biosynthesis pathway in G. max_{[PI 88788]} 3 dpi syncytia

Similar analyses for the 6 and 9 dpi time points allowed for the identification of other gene pathways that were amplified in G. Max_{[Peking/PI 548402]} as compared to G. max_{[PI 88788]}. Of note, the bras-sinosteroid pathway while still induced as compared to pericycle and surrounding cells, experiences suppression and attenuation in G. Max_{[Peking/PI 548402}] as compared to G. max_{[PI 88788]} at the 6 and 9 dpi time points. Other pathway analyses identified the biosynthesis of benzoxaz-noids (Cambier et al. 2001; Morant et al. 2008; Sasai et al. 2009), glycosphingolipids (Brodersen et al. 2002), and stilbenoid biosynthesis (Sobolev et al. 2010) to be induced and amplified in G. Max_{[Peking/PI 548402]} as compared to G. max_{[PI 88788]}. These pathways are important in defense of plants to pathogens, revealing important modes of defense that may be employed more efficiently by G. Max_{[Peking/PI 548402]} than G. max_{[PI 88788]}.

### CONCLUSION

It is highly likely that the resistance loci that have been mapped using the various soybean genotypes do not represent all of the genes involved in the process. Therefore, the mapped genes are an underestimate of the genes actually involved in the process. The analysis here employs an alternative strategy to identify genes that may be involved in the process for which no genetic lesions exist. The analysis presented here directly compares nematode feeding sites of the two major and different forms of the resistant reaction, G. Max_{[Peking/PI 548402]} (G. max_{[Peking]} type) and G. max_{[PI 88788]} (G. max_{[PI 88788]}-type). Specifically, the analyses explore the resistance processes of G. Max_{[Peking/PI 548402]} and G. max_{[PI 88788]}, the archetypal sources of nearly all of the resistance germplasm currently used in commercial production for resistance to H. glycines (Concibido et al. 2004; Colgrove and Niblack 2008). The resistant reaction, divided here into parasitism and resistance phases, allowed for a variety of comparative analyses to be performed that reveal gene expression that is unique to the two reaction types at the two phases of the resistant reaction. An aim of the gene expression experiments was to identify induced/amplified or suppressed/attenuated genes in each genotype during their respective resistant reactions. That objective was met. The expression analyses also identify genes that are common to G. Max_{[Peking/PI 548402]} and G. max_{[PI 88788]} that possibly relate to generalized features of the resistance process. The common gene expression features between the G. max_{[Peking/PI 548402]} and G. max_{[PI 88788]} reactions indicate that there is a conserved gene expression mechanism between the two reaction types. In cross-comparisons of the G. Max_{[Peking/PI 548402]} to the G. max_{[PI 88788]} resistant reaction types, the analyses identify a subset of genes that have induced and amplified levels of gene expression in either genotype at specific time points as well as those with differences throughout the resistant reaction. The genes identified in this and other related analyses (Klink et al. 2009a, 2010a) may actually associate with resistance in the G. Max_{[Peking/PI 548402]} and G. max_{[PI 88788]} reaction types, respectively. In some cases, these genes are found to have very large differences in relative amounts of gene expression. These genes may be a useful resource for association mapping of resistance genes found uniquely to G. Max_{[Peking/PI 548402]} and G. max_{[PI 88788]}. This is an important point because for decades it has been very difficult to identify the actual resistance genes not only because of the highly duplicated nature of the soybean genome, but because of localized duplications and deletions in and around resistance gene loci (Melito et al. 2010). The analyses demonstrate the value of using microarrays for related soybean genotypes undergoing nematode infection. Such analyses could be expanded to investigations of near isogenic lines (NILs) or recombinant inbred lines (RILs) for identifying candidate resistance genes. However, to determine the gene expression programs underlying the G. Max_{[Peking/PI 548402]} and G. max_{[PI 88788]} forms of the resistant reaction, it may be more appropriate to use the same nematode population to obtain each resistant reaction and cross compare them to their respective susceptible reactions by using a different nematode population (Klink et al. 2007a, 2009a, 2010b). In doing so, genotype-based differences that affect gene expression even in NILs and RILs are altogether avoided. The use of a high-throughput-Gateway® compatible transformation system designed specifically to investigate this system should aid in the identification of gene function during this important host-parasite interaction (Klink et al. 2009c).

The terms “comprising,” “including,” and “having,” as used in the claims and specification herein, shall be considered as indicating an open group that may include other elements not specified. The terms “a,” “an,” and the singular forms of words shall be taken to include the plural form of the same words, such that the terms mean that one or more of something is provided. The term “one” or “single” may be used to indicate that one and only one of something is intended. Similarly, other specific integer values, such as “two,” may be used when a specific number of things is intended. The terms “preferably,” “preferred,” “prefer,” “optionally,” “may,” and similar terms are used to indicate that an item, condition or step being referred to is an optional (not required) feature of the invention.

The invention has been described with reference to various specific and preferred embodiments and techniques. However, it should be understood that many variations and modifications may be made while remaining within the spirit and scope of the invention. It will be apparent to one of ordinary skill in the art that methods, devices, device elements, materials, procedures and techniques other than those specifically described herein can be applied to the practice of the invention as broadly disclosed herein without resort to undue experimentation. All art-known functional equivalents of methods, devices, device elements, materials, procedures and techniques described herein are intended to be encompassed by this invention. Whenever a range is disclosed, all subranges and individual values are intended to be encompassed. This invention is not to be limited by the embodiments disclosed, including any shown in the drawings or exemplified in the specification, which are given by way of example and not of limitation.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

### REFERENCES

All references throughout this application, for example patent documents including issued or granted patents or equivalents, patent application publications, and non-patent literature documents or other source material, are hereby incorporated by reference herein in their entireties, as though individually incorporated by reference, to the extent each reference is at least partially not inconsistent with the disclosure in the present application (for example, a reference that is partially inconsistent is incorporated by reference except for the partially inconsistent portion of the reference).
Abad P, Gouzy J, Aury J M, Castagnone-Sereno P, Danchin E G, Deleury E, Perfus-Barbeoch L, Anthouard V, Artiguenave F, Blok V C, Caillaud M C, Coutinho P M, Dasilva C, De Luca F, Deau F, Esquibet M, Flutre T, Goldstone J V, Hamamouch N, Hewezi T, Jaillon O, Jubin C, Leonetti P, Magliano M, Maier T R, Markov G V, McVeigh P, Pesole G, Poulain J, Robinson-Rechavi M, Sallet E, Se'gurens B, Steinbach D, Tytgat T, Ugarte E, van Ghelder C, Veronico P, Baum T J, Blaxter M, Bleve-Zacheo T, Davis E L, Ewbank J J, Favery B, Grenier E, Henrissat B, Jones J T, Laudet V, Maule A G, Quesneville H, Rosso M N, Schiex T, Smant G, Weissenbach J, Wincker P (2008) Genome sequence of the metazoan plant-parasitic nematode Meloidogyne incognita. Nat Biotechnol 26:909-915
Acido J R, Dropkin V H, Luedders V D (1984) Nematode population attrition and histopathology of Heterodera glycines-Soybean associations. J Nematol 16:48-57
Aist J R (1976) Papillae and related wound plugs of plant cells. Annu Rev Phytopathol 14:145-163
Alkharouf N W, Klink V P, Chouikha I B, Beard H S, MacDonald M H, Meyer S, Knap H T, Khan R, Matthews B F (2006) Timecourse microarray analyses reveals global changes in gene expression of susceptible Glycine max (soybean) roots during infection by Heterodera glycines (soybean cyst nematode). Planta 224: 838-852
Altschul S F, Madden T L, Schaffer A A, Zhang J, Zhang Z, Miller W, Lipman D J (1997) Gapped BLAST and PSI-BLAST: a new generation of protein database search programs. Nucleic Acids Res 25:3389-3402
Asano T, Masumura T, Kusano H, Kikuchi S, Kurita A, Shimada H, Kadowaki K (2002) Construction of a specialized cDNA library from plant cells isolated by laser capture microdissection: toward comprehensive analysis of the genes expressed in the rice phloem. Plant J 32:401-408
Bekal S, Craig J P, Hudson M E, Niblack T L, Domier L L, Lambert K N (2008) Genomic DNA sequence comparison between two inbred soybean cyst nematode biotypes facilitated by massively parallel 454 micro-bead sequencing. Mol Genet Genomics 279:535-543
Blee' E (2002) Impact of phyto-oxylipins in plant defense. Trends Plant Sci 7:315-322
Bostock R M, Kuc J, Laine R A (1981) Eicosapentaenoic and arachidonic acids from Phytophthora infestans elicit fungitoxic sesquiterpenes in the potato. Science 212:67-69
Bostock R M, Schaeffer D A, Hammerschmidt R (1986) Comparison of elicitor activities of arachidonic acid, fatty acids and glucans from Phytopthora infestans in hypersensitivity expression in potato tuber. Physiol Mol Plant Pathol 29:349-360
Brodersen P, Petersen M, Pike H M, Olszak B, Skov S, Odum N, Jørgensen L B, Brown R E, Mundy J (2002) Knockout of Arabidopsis accelerated-cell-death11 encoding a sphingosine transfer protein causes activation of programmed cell death and defense. Genes Dev 16:490-502
Cai D, Kleine M, Kifle S, Hans-Joachim H, Sandal N N, Marcker K A, Klein-Lankhorst R M, Salentijn E M J, Lange W, Stiekema W J, Wyss U, Grundler F M W, Jung C (1997) Positional cloning of a gene for nematode resistance in sugar beet. Science 275:832-834
Caldwell B E, Brim C A, Ross J P (1960) Inheritance of resistance of soybeans to the soybean cyst nematode, Heterodera glycines. Agron J 52:635-636
Cambier V, Hance T, De Hoffmann E (2001) Effects of 1,4-benzoxazin-3-one derivatives from maize on survival and fecundity of Metopolophium dirhodum (Walker) on artificial diet. J Chem Ecol 27:359-370
Chandra-Shekara A C, Venugopal S C, Barman S R, Kachroo A, Kachroo P (2007) Plastidial fatty acid levels regulate resistance gene-dependent defense signaling in Arabidopsis. Proc Natl Acad Sci USA 104:7277-7282
Chinchilla D, Zipfel C, Robatzek S, Kemmerling B, Nu\ddot{}rnberger T, Jones J D, Felix G, Boller T (2007) A flagellin-induced complex of the receptor FLS2 and BAK1 initiates plant defence. Nature 448:497-500
Cole R A (1984) Phenolic acids associated with the resistance of lettuce cultivars to the lettuce root aphid. Ann Appl Biol 105:129-145
Colebatch G, Kloska S, Trevaskis B, Freund S, Altmann T, Udvardi M K (2002) Novel aspects of symbiotic nitrogen fixation uncovered by transcript profiling with cDNA arrays. Mol Plant Microbe Interact 15411-15420
Colgrove A L, Niblack T L (2008) Correlation of female indices from virulence assays on inbred lines and field populations of Heterodera glycines. J Nematol 40:39-45
Concibido V C, Diers B W, Arelli P R (2004) A decade of QTL mapping for cyst nematode resistance in soybean. Crop Sci 44:1121-1131
Edens R M, Anand S C, Bolla R I (1995) Enzymes of the phenylpropanoid pathway in soybean infected with Meloiodiogyne incognita or Heterodera glycines. J Nematol 27:292-303
Emmert-Buck M R, Bonner R F, Smith P D, Chuaqui R F, Zhuang Z, Goldstein S R, Weiss R A, Liotta L A (1996) Laser capture microdissection. Science 274:998-1001
Endo B Y (1964) Penetration and development of Heterodera glycines in soybean roots and related and related anatomical changes. Phytopathology 54:79-88
Endo B Y (1965) Histological responses of resistant and susceptible soybean varieties, and backcross progeny to entry development of Heterodera glycines. Phytopathology 55:375-381
Endo B Y (1991) Ultrastructure of initial responses of resistant and susceptible soybean roots to infection by Heterodera glycines. Rev Nematol 14:73-94
Eulgem T, Somssich I E (2007) Networks of WRKY transcription factors in defense signaling. Curr Opin Plant Biol 10:366-371
Gao X, Starr J, Go\ddot{}bel C, Engelberth J, Feussner I, Tumlinson J, Kolomiets M (2008) Maize 9-lipoxygenase ZmLOX3 controls development, root-specific expression of defense genes, and resistance to root-knot nematodes. MPMI 21:98-109
Glover K D, Wang D, Arelli P R, Carlson S R, Cianzio S R, Diers B W (2004) Near isogenic lines confirm a soybean cyst nematode resistance gene from PI 88788 on linkage group. J Crop Sci 44:936-941
Golden A M, Epps J M, Riggs R D, Duclos L A, Fox J A, Bernard R L (1970) Terminology and identity of infraspecific forms of the soybean cyst nematode (Heterodera glycines). Plant Dis Rep 54:544-546
Halbrendt J, Lewis S, Shipe E (1992) A technique for evaluating Heterodera glycines development in susceptible and resistant soybean. J Nematol 24:84-91
Hardham A R, Takemoto D, White R G (2008) Rapid and dynamic subcellular reorganization following mechanical stimulation of Arabidopsis epidermal cells mimics responses to fungal and oomycete attack. BMC Plant Biol 8:63
Hartwig E E, Epps J M (1978) Registration of Bedford soybeans. Crop Sci 18:915
He K, Gou X, Yuan T, Lin H, Asami T, Yoshida S, Russell S D, Li J (2007) BAK1 and BKK1 regulate brassinosteroid-dependent growth and brassinosteroid-independent cell-death pathways. Curr Biol 17:1109-1115
Heese A, Hann D R, Gimenez-Ibanez S, Jones A M, He K, Li J, Schroeder J I, Peck S C, Rathjen J P (2007) The receptor-like kinase SERK3/BAK1 is a central regulator of innate immunity in plants. Proc Natl Acad Sci USA 104:12217-12222
Isenberg G, Bielser W, Meier-Ruge W, Remy E (1976) Cell surgery by laser microdissection: a preparative method. J Microsc 107: 19-24
Jones D A, Thomas C M, Hammond-Kosack K E, Balint-Kurti P J, Jones J D (1994) Isolation of the tomato Cf-9 gene for resistance to Cladosporium fulvum by transposon tagging. Science 266: 789-793
Kachroo A, Lapchyk L, Fukushige H, Hildebrand D, Klessig D, Kachroo P (2003) Plastidial fatty acid signaling modulates salicylic acid- and jasmonic acid-mediated defense pathways in the Arabidopsis ssi2 mutant. Plant Cell 15:2952-2965
Kachroo A, Fu D Q, Havens W, Navarre D, Kachroo P, Ghabrial S A (2008) An oleic acid-mediated pathway induces constitutive defense signaling and enhanced resistance to multiple pathogens in soybean. Mol Plant Microbe Interact 21:564-575
Kim K S, Riggs R D (1992) Cytopathological reactions of resistant soybean plants to nematode invasion. In: Wrather J A, Riggs R D (eds) Biology and management of the soybean cyst nematode. APS Press, St. Paul, pp 157-168
Kim Y H, Riggs R D, Kim K S (1987) Structural changes associated with resistance of soybean to Heterodera glycines. J Nematol 19:177-187
Klink V P, MacDonald M, Alkharouf N, Matthews B F (2005) Laser capture microdissection (LCM) and expression analyses of Glycine max (soybean) syncytium containing root regions formed by the plant pathogen Heterodera glycines (soybean cyst nematode). Plant Mol Biol 59:969-983
Klink V P, Overall C C, Alkharouf N, MacDonald M H, Matthews B F (2007a) Laser capture microdissection (LCM) and comparative microarray expression analysis of syncytial cells isolated from incompatible and compatible soybean roots infected by soybean cyst nematode (Heterodera glycines). Planta 226:1389-1409
Klink V P, Overall C C, Alkharouf N, MacDonald M H, Matthews B F (2007b) A comparative microarray analysis of an incompatible and compatible disease response by soybean (Glycine max) to soybean cyst nematode (Heterodera glycines) infection. Planta 226:1423-1447
Klink V P, MacDonald M H, Martins V E, Park S-C, Kim K-H, Baek S-H, Matthews B F (2008) MiniMax, a new diminutive Glycine max variety, with a rapid life cycle, embryogenic potential and transformation capabilities. Plant Cell Tissue Organ Cult 92: 183-195
Klink V P, Hosseini P, Matsye P, Alkharouf N, Matthews B F (2009a) A gene expression analysis of syncytia laser microdissected from the roots of the Glycine max (soybean) genotype PI 548402 (Peking) undergoing a resistant reaction after infection by Heterodera glycines (soybean cyst nematode). Plant Mol Biol 71:525-567
Klink V P, Hosseini P, MacDonald M H, Alkharouf N, Matthews B F (2009b) Population-specific gene expression in the plant pathogenic nematode Heterodera glycines exists prior to infection and during the onset of a resistant or susceptible reaction in the roots of the Glycine max genotype Peking. BMC Genomics 10:111
Klink V P, Kim K-H, Martins V E, MacDonald M H, Beard H S, Alkharouf N W, Park S-C, Matthews B F (2009c) A correlation between host-mediated expression of parasite genes as tandem inverted repeats and abrogation of the formation of female Heterodera glycines cysts during infection of Glycine max. Planta 230:53-71
Klink V P, Hosseini P, Matsye P, Alkharouf N, Matthews B F (2010a) Syncytium gene expression in Glycine max[PI 88788] roots undergoing a resistant reaction to the parasitic nematode Heterodera glycines. Plant Physiol Biochem 48:176-193
Klink V P, Overall C C, Alkharouf N, MacDonald M H, Matthews B F (2010b) Microarray detection calls as a means to compare transcripts expressed within syncytial cells isolated from incompatible and compatible soybean (Glycine max) roots infected by the soybean cyst nematode (Heterodera glycines). J Biomed Biotechnol 2010:491217 (1-30)
Kosma D K, Nemacheck J A, Jenks M A, Williams C E (2010) Changes in properties of wheat leaf cuticle during interactions with Hessian fly. Plant J 63:31-43 Lauritis J A, Rebois R, Graney L S (1983) Development of Heterodera glycines Ichinohe on soybean, Glycine max (L.) Merr., under gnotobiotic conditions. J Nematol 15:272-280
Leszczynski B, Wright L C, Bakowski T (1989) Effect of secondary plant substances on winter wheat resistance to grain aphid. Entomol Exp Appl 52:135-139
Lu D, Wu S, Gao X, Zhang Y, Shan L, He P (2010) A receptor-like cytoplasmic kinase, BIK1, associates with a flagellin receptor complex to initiate plant innate immunity. Proc Natl Acad Sci USA 107:496-501
Mahalingham R, Skorupska H T (1996) Cytological expression of early response to infection by Heterodera glycines Ichinohe in resistant PI 437654 soybean. Genome 39:986-998
Mahalingham R, Wang G, Knap H T (1999) Polygalacturonase and polygalacturonase inhibitor protein: gene isolation and transcription in Glycine max-Heterodera glycines interactions. Mol Plant Microbe Interact 12:490-498
Matson A L, Williams L F (1965) Evidence of a fourth gene for resistance to the soybean cyst nematode. Crop Sci 5:477
McElver J, Tzafrir I, Aux G, Rogers R, Ashby C, Smith K, Thomas C, Schetter A, Zhou Q, Cushman M A, Tossberg J, Nickle T, Levin J Z, Law M, Meinke D, Patton D (2001) Insertional mutagenesis of genes required for seed development in Arabidopsis thaliana. Genetics 159:1751-1763
Meier-Ruge W, Bielser W, Remy E, Hillenkamp F, Nitsche R, Unsold R (1976) The laser in the Lowry technique for microdissection of freeze-dried tissue slices. Histochem J 8:387-401
Melito S, Heuberger A L, Cook D, Diers B W, MacGuidwin A E, Bent A F (2010) A nematode demographics assay in transgenic roots reveals no significant impacts of the Rhg1locus LRR-kinase on soybean cyst nematode resistance. BMC Plant Biol 10:104
Milligan S B, Bodeau J, Yaghoobi J, Kaloshian I, Zabel P, Williamson V M (1998) The root knot nematode resistance gene Mi from tomato is a member of the leucine zipper, nucleotide binding, leucine-rich repeat family of plant genes. Plant Cell 10:1307-1319
Morant A V, Jorgensen K, Jorgensen C, Paquette S M, Sa'nchez-Pe'rez R, Møller B L, Bak S (2008) Beta-glucosidases as detonators of plant chemical defense. Phytochemistry 69:1795-1813
Nakashita H, Yasuda M, Nitta T, Asami T, Fujioka S, Arai Y, Sekimata K, Takatsuto S, Yamaguchi I, Yoshida S (2003) Brassinosteroid functions in a broad range of disease resistance in tobacco and rice. Plant J 33:887-898
Niblack T L, Arelli P R, Noel G R, Opperman C H, Orf J H, Schmitt D P, Shannon J G, Tylka G L (2002) A revised classification scheme for genetically diverse populations of Heterodera glycines. J Nematol 34:279-288
Popescu S C, Popescu G V, Bachan S, Zhang Z, Gerstein M, Snyder
M, Dinesh-Kumar S P (2008) MAPK target networks in Arabidopsis thaliana revealed using functional protein microarrays. Genes Dev 23:80-92
Rao-Arelli A P (1994) Inheritance of resistance to Heterodera glycines race 3 in soybean accessions. Plant Dis 78:898-900
Reina-Pinto J J, Voisin D, Kurdyukov S, Faust A, Haslam R P, Michaelson L V, Efremova N, Franke B, Schreiber L, Napier J A, Yephremov A (2009) Misexpression of FATTY ACID ELONGATION1 in the Arabidopsis epidermis induces cell death and suggests a critical role for phospholipase A2 in this process. Plant Cell 21:1252-1272
Ricker K E, Bostock R M (1992) Evidence for release of the elicitor arachidonic acid and its metabolites from sporangia of Phytophthora infestans during infection of potato. Physiol Mol Plant Pathol 41:61-72
Riggs R D (1988) Races of Heterodera glycines. Nematropica 18:163-170
Riggs R D (1992) Chapter 10: Host range. In: Riggs R D, Wrather J A (eds) Biology and management of the soybean cyst nematode. APS Press, St Paul, pp 107-114
Riggs R D, Schmitt D P (1988) Complete characterization of the race scheme for Heterodera glycines. J Nematol 20:392-395
Riggs R D, Schmitt D P (1991) Optimization of the Heterodera glycines race test procedure. J Nematol 23:149-154
Riggs R D, Kim K S, Gipson I (1973) Ultrastructural changes in Peking soybeans infected with Heterodera glycines. Phytopathology 63:76-84
Ross J P (1958) Host-Parasite relationship of the soybean cyst nematode in resistant soybean roots. Phytopathology 48:578-579
Ross J P (1962) Physiological strains of Heterodera glycines. Plant Dis Rep 46:766-769
Sardanelli S, Kenworthy W J (1997) Soil moisture control and direct seeding for bioassay of Heterodera glycines on soybean. J Nematol 29(suppl):625-634
Sasai H, Ishida M, Murakami K, Tadokoro N, Ishihara A, Nishida R, Mori N (2009) Species-specific glucosylation of DIMBOA in larvae of the rice Armyworm. Biosci Biotechnol Biochem 73:1333-1338
Sass J E (1958) Botanical microtechnique. Iowa State College Press, Ames
Sasser J N, Freckman D W (1987) A world perspective on nematology: the role of the society. In: Veech J A, Dickerson D W (eds) Vistas on nematology. Society of Nematologists, Hyattsville, pp 7-14
Scheideler M, Schlaich N L, Fellenberg K, Beissbarth T, Hauser N C, Vingron M, Slusarenko A J, Hoheisel J D (2002) Monitoring the switch from housekeeping to pathogen defense metabolism in Arabidopsis thaliana using cDNA arrays. J Biol Chem 277: 10555-105561
Schmelzer E (2002) Cell polarization, a crucial process in fungal defence. Trends Plant Sci 7:411-415
Shannon J G, Arelli P R, Young L D (2004) Breeding for resistance and tolerance. In: Schmitt D P, Wrather J A, Riggs R D (eds) Biology and management of soybean cyst nematode, 2nd edn. Schmitt & Associates of Marceline, Marceline, pp 155-180
Sobolev V S, Neff S A, Gloer J B (2010) New dimeric stilbenoids from fungal-challenged peanut (Arachis hypogaea) seeds. J Agric Food Chem 58:875-881 The Gene Ontology Consortium (2004) The gene ontology (G O) database and informatics resource. Nucleic Acids Res 32:D258-D261
Tzafrir I, Pena-Muralla R, Dickerman A, Berg M, Rogers R, Hutchens S, Sweeney T C, McElver J, Aux G, Patton D, Meinke D (2001) Identification of genes required for embryo development in Arabidopsis. Plant Physiol 135:1206-1220
Wang Y, Cai Q N, Zhang Q W, Han Y (2006) Effect of the secondary substances from wheat on the growth and digestive physiology of cotton bollworm Helicoverpa armigera (Lepidoptera: Noctuidae). Eur J Entomol 103:255-258
Weyman P D, Pan Z, Feng Q, Gilchrist D G, Bostock R M (2006a) A circadian rhythm-regulated tomato gene is induced by Arachidonic acid and Phytophthora infestans infection. Plant Physiol 140:235-248 Weyman P D, Pan Z, Feng Q, Gilchrist D G, Bostock R M (2006b) DEA1, a circadian- and cold-regulated tomato gene, protects yeast cells from freezing death. Plant Mol Biol 62:547-559
Wrather J A, Koenning S R (2006) Estimates of disease effects on soybean yields in the United States 2003-2005. J Nematol 38:173-180
Wrather J A, Stienstra W C, Koenning S R (2001) Soybean disease loss estimates for the United States from 1996 to 1998. Can J Plant Pathol 23:122-131

We claim:

1. A method for identifying nematode resistant genes in Glycine max comprising:
a. performing a microarray analyses comparing these cytologically and developmentally distinct resistant reactions;
b. observing differences in gene expression in pericycle and surrounding cells even before infection.
c. performing gene pathway analyses that compare the two genotypes before, at various times during, constitutively throughout the resistant reaction, and at all time points prior to and during the resistant reaction.
